(19) United States
(12) Patent Application Publication
(10) Pub. No.: US 2025/0339214 A1
(43) Pub. Date: Nov. 6, 2025

(54) METHODS AND SYSTEMS FOR AUTOMATICALLY GENERATING A SURGICAL OPERATIVE NOTE

(71) Applicant: Proprio, Inc., Seattle, WA (US)

(72) Inventors: Adam Gabriel Jones, Seattle, WA (US); Thomas A. Carls, Seattle, WA (US); Neeraj Mainkar, Baltimore, MD (US); Jocelyn Elaine Barker, San Jose, CA (US); Kabir S Gulati, San Francisco, CA (US)

(21) Appl. No.: 19/197,838

(22) Filed: May 2, 2025

Related U.S. Application Data
(60) Provisional application No. 63/642,440, filed on May 3, 2024.
(60) Provisional application No. 63/692,031, filed on Sep. 7, 2024.

(51) Int. Cl. A61B 34/00; A61B 90/00; G06F 40/134; G06F 40/40; G06V 10/44; G06V 20/40; G06V 20/50; G16H 15/00

(52) U.S. Cl. CPC A61B 34/25; A61B 90/361; G06F 40/134; G06F 40/40; G06V 10/44; G06V 20/46; G06V 20/50; G16H 15/00; A61B 2034/256; G06V 2201/033

(57) ABSTRACT

Methods of generating a surgical operative note for a surgical procedure, such as a spinal surgical procedure, and associated systems and devices are disclosed herein. In some embodiments, a representative method includes capturing surgical procedure data of the surgical procedure with a sensor array positioned to view the surgical procedure, and identifying features in the surgical procedure data relevant to the surgical procedure. The method can further comprise processing the identified features to provide contextual information about the surgical procedure, and utilizing an artificial intelligence (AI) application to generate the operative note based on the identified features and the contextual information. The operative note can include a natural language, structured, and coherent description of the surgical procedure that summarizes the surgical procedure, including the type of surgery performed, specific surgical techniques used, intraoperative findings, and/or postoperative care instructions.

### CROSS-REFERENCE TO RELATED APPLICATIONS

This application claims the benefit of (i) U.S. Provisional Patent Application No. 63/692,031, filed Sep. 7, 2024, and titled “METHODS AND SYSTEMS FOR AUTOMATICALLY GENERATING A SURGICAL OPERATIVE NOTE,” and (ii) U.S. Provisional Patent Application No. 63/642,440, filed May 3, 2024, and titled “METHODS AND SYSTEMS FOR AUTOMATICALLY GENERATING A SURGICAL OPERATIVE NOTE,” each of which is incorporated herein by reference in its entirety.

### TECHNICAL FIELD

The present technology generally relates to methods, systems, and devices for automatically generating a surgical operative note documenting a surgical procedure, such as a spinal surgical procedure, based at least in part on data captured intraoperatively by a sensor array.

### BACKGROUND

A surgical operative note, also known as an operative report or operative record, is a medical document that serves several important purposes in the context of surgical procedures. For example, the primary purpose of a surgical operative note is to provide a detailed record of the surgical procedure performed. It includes essential information, such as the date and time of the surgery, the name of the surgeon and surgical team members, the type of procedure performed, and a step-by-step description of the surgical techniques used. Operative notes also serve as a means of communication between members of the healthcare team, including surgeons, nurses, anesthesiologists, and other healthcare providers involved in the patient's care. It ensures that all team members have access to accurate and up-to-date information about the surgical procedure and any intraoperative findings or complications.

Operative notes also serve to meet legal and regulatory requirements. For example, operative notes are legal documents that are part of the patient's medical record. They provide a legal record of the surgical procedure performed, including any preoperative assessments, intraoperative interventions, and postoperative care provided. Accurate and comprehensive documentation is essential for meeting regulatory requirements and potential medicolegal issues.

Operative notes also play a vital role in the billing process for surgical procedures in healthcare settings. For example, healthcare providers can use the detailed information in an operative note about the surgical procedure performed (e.g., the type of surgery, specific surgical techniques used, any additional procedures or interventions performed, any complications encountered, etc.) to assign appropriate procedure codes to accurately describe the services rendered during the surgery. In addition to procedure codes, the operative note also includes information about the patient's diagnosis or medical condition necessitating the surgery. This information helps link the surgical procedure to the appropriate diagnosis code, which is used to justify the medical necessity of the surgery for billing and reimbursement purposes. Operative notes also document important details about the time complexity of a surgical procedure, such as the date and time of the surgery, the duration of the procedure, and any intraoperative findings or complications encountered. This information helps support the level of complexity and resources required for the surgery, which may influence reimbursement rates. Moreover, accurate and comprehensive documentation in the operative note is essential for compliance with billing and coding guidelines set forth by regulatory authorities, such as the Centers for Medicare and Medicaid Services (CMS) in the United States. Proper documentation ensures that billing claims meet the required standards for reimbursement and reduces the risk of audits or denials. The information documented in the operative notes also serves as the basis for determining reimbursement for the surgical procedure. Insurance payers, including government payers (e.g., Medicare, Medicaid) and private health insurers, review the surgical note to verify the medical necessity of the procedure, ensure appropriate coding, and calculate the reimbursement amount based on established fee schedules or reimbursement rates. Finally, in cases where billing claims are denied or audited, the operative note serves as the primary source of documentation to support the services billed. Healthcare providers may use the information in the operative note to appeal denials or respond to audit inquiries by providing additional documentation and justification for the billed services.

Operative notes are also valuable educational resources for medical students, residents, and other healthcare professionals learning about surgical techniques and procedures. They provide detailed descriptions of surgical techniques, anatomical landmarks, and intraoperative considerations that can help trainees understand the intricacies of surgical practice. Similarly, operative notes can be used for research purposes and quality improvement initiatives aimed at enhancing patient outcomes and surgical practice. Analysis of operative notes can identify trends, patterns, and areas for improvement in surgical techniques, patient care practices, and clinical outcomes.

Lastly, the information documented in operative notes is essential for providing follow-up care and monitoring patients' postoperative progress. It provides a reference for assessing the success of the surgical procedure, monitoring for complications, and guiding ongoing management and treatment decisions.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIG. 2 is a perspective view of an environment employing the imaging system of FIG. 1 in accordance with embodiments of the present technology.

FIG. 3 is an isometric view of a portion of the imaging system of FIG. 1 illustrating four cameras of a sensor array of the imaging system in accordance with embodiments of the present technology.

FIG. 4 is a block diagram of an operative note processing device in accordance with embodiments of the present technology.

FIG. 5 is an example of an operative note generated by the operative note processing device of FIG. 4 and that can be displayed on a display screen, computing device, user interface, and/or the like in accordance with embodiments of the present technology.

FIG. 6A is an example of the operative note of FIG. 5 enhanced with embedded via the operative note processing device of FIG. 4 in accordance with embodiments of the present technology.

FIGS. 6B-6E are examples of the enhanced operative note of FIG. 6A after a user selection of different ones of the hyperlinks in accordance with embodiments of the present technology.

FIGS. 7A-7D are examples the enhanced operative note of FIG. 6A including the solicitation of user feedback in accordance with embodiments of the present technology.

FIG. 8 is a flow diagram of a process or method carried out by the operative note processing device of FIG. 4 for automatically generating a surgical operative note in accordance with embodiments of the present technology.

FIG. 9 is a block diagram of a functional computing environment in which at least some operations described herein can be implemented in accordance with embodiments of the present technology.

FIG. 10 is a block diagram that illustrates an example of a computer system in which at least some operations described herein can be implemented in accordance with embodiments of the present technology.

FIG. 11 is a block diagram of an example transformer.

FIG. 12 is a block diagram illustrating an architecture for large language model applications, according to some implementations.

### DETAILED DESCRIPTION

Aspects of the present technology are directed generally to methods of generating a surgical operative note for a surgical procedure, such as a spinal surgical procedure, and associated systems and devices. In some embodiments, a representative method includes acquiring surgical procedure data of the surgical procedure and identifying features in the surgical procedure data relevant to the surgical procedure. The method can further comprise processing the identified features to provide contextual information about the surgical procedure, and utilizing an artificial intelligence (AI) application to generate the operative note based on the identified features and the contextual information. The operative note can include a natural language, structured, and coherent description of the surgical procedure that summarizes the surgical procedure, including the type of surgery performed, specific surgical techniques used, intraoperative findings, postoperative care instructions, and/or the like.

In some embodiments, the surgical procedure data includes include multi-modal data including image, video, text, and/or other data captured intraoperatively (e.g., by a sensor array positioned to view the surgical procedure) and/or preoperatively (e.g., preoperative computed tomography (CT) and/or magnetic resonance imaging (MRI) images). The surgical procedure data can be acquired in real time or near real time during the surgical procedure, or can be received in full after completion of the surgical procedure.

In some embodiments, identifying the relevant features in the surgical procedure data includes utilizing computer vision techniques such as object detection, motion tracking, and/or image segmentation to identify and extract surgical actions (e.g., blunt dissection, deep dissection, incision, closure, laminotomy), anatomical landmarks (e.g., spinous processes, inter-spinous ligaments, lamina, pars and facets), instrument movements (e.g., pedicle screw entry, cutting instrument usage, retractor usage), and intraoperative events (e.g., incision, dissection, closure). In some embodiments, processing the identified features to provide contextual information about the surgical procedure can include integrating the identified features from multiple data modalities to provide context and temporal understanding of the surgical procedure. For example, the same features of the surgical procedure identified in different data modalities can be grouped together to provide a temporal understanding of the surgical procedure. Additionally, one or more AI applications can be used to provide the contextual information about the features relevant to the surgical procedure.

In some embodiments, the method further includes embedding hyperlinks and/or other indicia into the operative note that link textual descriptions in the operative note to corresponding identified features in the surgical procedure data. The hyperlinks can allow a user viewing the operative note on a user interface (e.g., a computing device) to quickly retrieve surgical procedure data (e.g., a video segment or image) corresponding to certain textual descriptions in the operative note.

In some embodiments, the method further includes validating the accuracy and completeness of the operative note through automated checks. The operative note can be updated automatically to correct for any inaccuracies and/or to fill in omitted information. The method can also include validating the accuracy and completeness of the operative by soliciting user feedback. For example, the method can include inserting feedback indicators into the operative note and that can be selected by a user (e.g., a surgeon and/or surgical team member) viewing the operative noted on a user interface to confirm or deny the accuracy of textual descriptions in the operative note. Any inaccuracies and/or omissions in the operative note can be corrected by the user. In some embodiments, the updates to the operative note made automatically and/or by the user can be used as part of a reinforcement learning algorithm to update the model(s) used by the AI application.

In some embodiments, the method further includes providing the operative note to one or more requestors. For example, the method can include providing the operative note to one or more (i) clinical health care systems for continued patient care, learning, training, etc., (ii) financial systems for verifying the medical necessity of the surgical procedure, ensuring appropriate coding, calculating the reimbursement amount based on established fee schedules or reimbursement rates, etc., and/or (iii) other interested parties (e.g., third party systems and/or applications).

In some aspects of the present technology, the methods, systems, and devices of the present technology can automatically generate an accurate surgical operative note describing a surgical procedure in a manner that provides improved efficiency, accuracy, standardization, and documentation compared to conventional manual methods for preparing operative notes. Regarding efficiency, the present technology can improve efficiency by automatically generating operative notes with no, reduced, and/or minimal effort on the part of a user (e.g., a surgeon or surgical team member). That is, the user need not manually prepare an operative note postoperatively and, at most, can simply provide select feedback to verify the accuracy of an automatically-generated operative note and/or to fill in any omissions therein. Regarding accuracy, the present technology can leverage AI algorithms and surgical data (e.g., video data) to produce accurate and detailed operative notes with minimal human intervention. Regarding standardization, the present technology can promote consistency and standardization in operative note documentation across surgical procedures and healthcare providers. Finally, regarding documentation, the present technology can capture rich, hyperlinked, and comprehensive information from surgical videos and other surgical procedure data, enhancing the quality and completeness of operative notes for clinical and medico-legal purposes. Accordingly, the present technology offers significant benefits in terms of efficiency, accuracy, standardization, and documentation, ultimately improving patient care and clinical workflow in surgical settings.

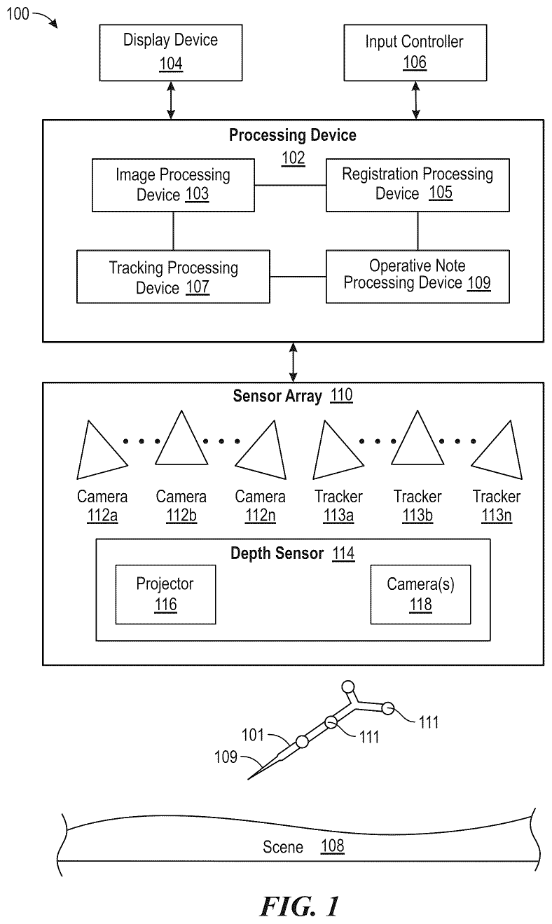

Specific details of several embodiments of the present technology are described herein with reference to FIGS. 1-11. The present technology, however, can be practiced without some of these specific details. In some instances, well-known structures and techniques often associated with sensor arrays, RGB imaging, depth sensing, machine learning and artificial intelligence (AI) processes/algorithms/models, registration processes, and the like have not been shown in detail so as not to obscure the present technology.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section. Moreover, although frequently described in the context of generating an operative note for a spinal surgical procedure, the present technology can be used to automatically generate operative notes for other types of surgical procedures, such as general surgical procedures, orthopedic surgical procedures, neurosurgical procedures, laparoscopic procedures, etc.

The accompanying Figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

The headings provided herein are for convenience only and should not be construed as limiting the subject matter disclosed. To the extent any materials incorporated herein by reference conflict with the present disclosure, the present disclosure controls.

### I. Selected Embodiments of Imaging Systems

FIG. 1 is a schematic view of an imaging system 100 (“system 100”) in accordance with embodiments of the present technology. In some embodiments, the system 100 can be a synthetic augmented reality system, a virtual-reality imaging system, an augmented-reality imaging system, a mediated-reality imaging system, and/or a non-immersive computational imaging system. In the illustrated embodiment, the system 100 includes a processing device 102 that is communicatively coupled to one or more display devices 104, one or more input controllers 106, and a sensor array 110 (e.g., a camera array, a sensor head, and/or the like). In other embodiments, the system 100 can comprise additional, fewer, or different components. In some embodiments, the system 100 includes some features that are generally similar or identical to those of the mediated-reality imaging systems disclosed in (i) U.S. patent application Ser. No. 16/586,375, filed Sep. 27, 2019, titled “CAMERA ARRAY FOR A MEDIATED-REALITY SYSTEM,” and/or (ii) U.S. patent application Ser. No. 15/930,305, filed May 12, 2020, and titled “METHODS AND SYSTEMS FOR IMAGING A SCENE, SUCH AS A MEDICAL SCENE, AND TRACKING OBJECTS WITHIN THE SCENE,” each of which is incorporated herein by reference in its entirety.

In the illustrated embodiment, the sensor array 110 includes a plurality of cameras 112 (identified individually as cameras 112a-n; which can also be referred to as first cameras) that can each capture images of a scene 108 (e.g., first image data) from a different perspective. The scene 108 can include for example, a patient undergoing surgery (e.g., spinal surgery) and/or another medical procedure. In other embodiments, the scene 108 can be another type of scene. The sensor array 110 can further include dedicated object tracking hardware 113 (e.g., including individually identified trackers 113a-n) that captures positional data of one more objects, such as an instrument 101 (e.g., a surgical instrument or tool) having a tip 119, to track the movement and/or orientation of the objects through/in the scene 108. In some embodiments, the cameras 112 and the trackers 113 are positioned at fixed locations and orientations (e.g., poses) relative to one another. For example, the cameras 112 and the trackers 113 can be structurally secured by/to a mounting structure (e.g., a common frame) at predefined fixed locations and orientations. In some embodiments, the cameras 112 are positioned such that neighboring cameras 112 share overlapping views of the scene 108. In general, the position of the cameras 112 can be selected to maximize clear and accurate capture of all or a selected portion of the scene 108. Likewise, the trackers 113 can be positioned such that neighboring trackers 113 share overlapping views of the scene 108. Therefore, all or a subset of the cameras 112 and the trackers 113 can have different extrinsic parameters, such as position and orientation (e.g., pose).

In some embodiments, the cameras 112 in the sensor array 110 are synchronized to capture images of the scene 108 simultaneously (within a threshold temporal error). In some embodiments, all or a subset of the cameras 112 are light field, plenoptic, and/or RGB cameras that capture information about the light field emanating from the scene 108 (e.g., information about the intensity of light rays in the scene 108 and also information about a direction the light rays are traveling through space). In some embodiments, image data from the cameras 112 can be used to reconstruct a light field of the scene 108. More specifically, the cameras 112 can be RGB cameras that capture a combined image data set for reconstructing a light field of the scene 108. Therefore, in some embodiments the images captured by the cameras 112 encode depth information representing a surface geometry of the scene 108. In some embodiments, the cameras 112 are substantially identical. In other embodiments, the cameras 112 include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses. For example, the cameras 112 can be separate light field cameras each having their own image sensors and optics. In other embodiments, some or all of the cameras 112 can comprise separate microlenslets (e.g., lenslets, lenses, microlenses) of a microlens array (MLA) that share a common image sensor. In other embodiments, some or all of the cameras 112 can be RGB (e.g., color) cameras having visible imaging sensors that together provide a light field data set of the scene 108.

In some embodiments, the trackers 113 are imaging devices, such as infrared (IR) cameras that can capture images of the scene 108 from a different perspective compared to other ones of the trackers 113. Accordingly, the trackers 113 and the cameras 112 can have different spectral sensitives (e.g., infrared vs. visible wavelength). In some embodiments, the trackers 113 capture image data of a plurality of optical markers (e.g., fiducial markers, marker balls) in the scene 108, such as markers 111 coupled to the instrument 101.

In the illustrated embodiment, the sensor array 110 further includes a depth sensor 114. In some embodiments, the depth sensor 114 includes (i) one or more projectors 116 that project a structured light pattern onto/into the scene 108 and (ii) one or more depth cameras 118 (which can also be referred to as second cameras) that capture second image data of the scene 108 including the structured light projected onto the scene 108 by the projector 116. The projector 116 can project a speckled pattern or a pattern of dots, for example. The projector 116 and the depth cameras 118 can operate in the same wavelength and, in some embodiments, can operate in a wavelength different than the cameras 112. For example, the cameras 112 can capture the first image data in the visible spectrum, while the depth cameras 118 capture the second image data in the infrared spectrum. In some embodiments, the depth cameras 118 have a resolution that is less than a resolution of the cameras 112. For example, the depth cameras 118 can have a resolution that is less than 70%, 60%, 50%, 40%, 30%, or 20% of the resolution of the cameras 112. In other embodiments, the depth sensor 114 can include other types of dedicated depth detection hardware (e.g., a LiDAR detector) for determining the surface geometry of the scene 108. In other embodiments, the sensor array 110 can omit the projector 116 and/or the depth cameras 118.

In the illustrated embodiment, the processing device 102 includes an image processing device 103 (e.g., an image processor, an image processing module, an image processing unit), a registration processing device 105 (e.g., a registration processor, a registration processing module, a registration processing unit), a tracking processing device 107 (e.g., a tracking processor, a tracking processing module, a tracking processing unit), and a operative note processing device 109 (e.g., a operative note processor, an operative note processing module, an operative note processing unit, an operative note generation device). The image processing device 103 can (i) receive the first image data captured by the cameras 112 (e.g., light field images, light field image data, RGB images) and depth information from the depth sensor 114 (e.g., the second image data captured by the depth cameras 118), and (ii) process the image data and depth information to synthesize (e.g., generate, reconstruct, render) a three-dimensional (3D) output image of the scene 108 corresponding to a virtual camera perspective (e.g., a novel camera perspective). The output image can correspond to an approximation of an image of the scene 108 that would be captured by a camera placed at an arbitrary position and orientation corresponding to the virtual camera perspective. In some embodiments, the image processing device 103 can further receive and/or store calibration data for the cameras 112 and/or the depth cameras 118 and synthesize the output image based on the image data, the depth information, and/or the calibration data. More specifically, the depth information and the calibration data can be used/combined with the images from the cameras 112 to synthesize the output image as a 3D (or stereoscopic 2D) rendering of the scene 108 as viewed from the virtual camera perspective.

In some embodiments, the image processing device 103 can synthesize the output image using any of the methods disclosed in U.S. patent application Ser. No. 16/457,780, filed Jun. 28, 2019, and titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” which is incorporated herein by reference in its entirety. In other embodiments, the image processing device 103 can generate the virtual camera perspective based only on the images captured by the cameras 112—without utilizing depth information from the depth sensor 114. For example, the image processing device 103 can generate the virtual camera perspective by interpolating between the different images captured by one or more of the cameras 112. In some embodiments, the image processing device 103 utilizes a neural radiance field (NeRF) rendering algorithm to synthesize and render an output image of the scene 108 based on RGB images captured by the cameras 112 and depth data captured by the depth sensor 114.

The image processing device 103 can synthesize the output image from images captured by a subset (e.g., two or more) of the cameras 112 in the sensor array 110, and does not necessarily utilize images from all of the cameras 112. For example, for a given virtual camera perspective, the processing device 102 can select a stereoscopic pair of images from two of the cameras 112. In some embodiments, such a stereoscopic pair can be selected to be positioned and oriented to most closely match the virtual camera perspective. In some embodiments, the image processing device 103 (and/or the depth sensor 114) estimates a depth for each surface point of the scene 108 relative to a common origin to generate a point cloud and/or a 3D mesh that represents the surface geometry of the scene 108. Such a representation of the surface geometry can be referred to as a surface reconstruction, a 3D reconstruction, a 3D surface reconstruction, a depth map, a depth surface, and/or the like. In some embodiments, the depth cameras 118 of the depth sensor 114 detect the structured light projected onto the scene 108 by the projector 116 to estimate depth information of the scene 108. In some embodiments, the image processing device 103 estimates depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 114. In other embodiments, depth may be acquired by a specialized set of the cameras 112 performing the aforementioned methods in another wavelength. In some embodiments, the image processing device 103 can generate a stereoscopic view by selecting images from a pair of the cameras 112 using any of the methods disclosed in U.S. patent application Ser. No. 17/521,235, filed Nov. 11, 2021, and titled “METHODS FOR GENERATING STEREOSCOPIC VIEWS IN MULTICAMERA SYSTEMS, AND ASSOCIATED DEVICES AND SYSTEMS,” which is incorporated herein by reference in its entirety.

In some embodiments, the registration processing device 105 receives and/or stores initial image data, such as image data of a three-dimensional volume of a patient (3D image data). The image data can include, for example, computerized tomography (CT) scan data, magnetic resonance imaging (MRI) scan data, ultrasound images, fluoroscope images, and/or other medical or other image data. The image data can be segmented or unsegmented. The registration processing device 105 can register the initial image data to the real time images captured by the cameras 112 and/or the depth sensor 114 by, for example, determining one or more transforms/transformations/mappings between the two. The processing device 102 (e.g., the image processing device 103) can then apply the one or more transformations to the initial image data such that the initial image data can be aligned with (e.g., overlaid on) the output image of the scene 108 in real time or near real time on a frame-by-frame basis, even as the virtual perspective changes. That is, the image processing device 103 can fuse the initial image data with the real time output image of the scene 108 to present a mediated-reality view that enables, for example, a surgeon to simultaneously view a surgical site in the scene 108 and the underlying 3D anatomy of a patient undergoing an operation. In some embodiments, the registration processing device 105 can register the initial image data to the real time images by using any of the methods disclosed in U.S. patent application Ser. No. 17/140,885, filed Jan. 4, 2021, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and/or U.S. patent application Ser. No. 18/084,389, filed Dec. 19, 2022, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” each of which is incorporated by reference herein in its entirety.

In some embodiments, the tracking processing device 107 processes positional data captured by the trackers 113 to track objects (e.g., the instrument 101) within the vicinity of the scene 108. For example, the tracking processing device 107 can determine the position of the markers 111 in the 2D images captured by two or more of the trackers 113, and can compute the 3D position of the markers 111 via triangulation of the 2D positional data. More specifically, in some embodiments the trackers 113 include dedicated processing hardware for determining positional data from captured images, such as a centroid of the markers 111 in the captured images. The trackers 113 can then transmit the positional data to the tracking processing device 107 for determining the 3D position of the markers 111. In other embodiments, the tracking processing device 107 can receive the raw image data from the trackers 113. In a surgical application, for example, the tracked object can comprise a surgical instrument, an implant, a hand or arm of a physician or assistant, and/or another object having the markers 111 mounted thereto. In some embodiments, the processing device 102 can recognize the tracked object as being separate from the scene 108, and can apply a visual effect to the 3D output image to distinguish the tracked object by, for example, highlighting the object, labeling the object, and/or applying a transparency to the object.

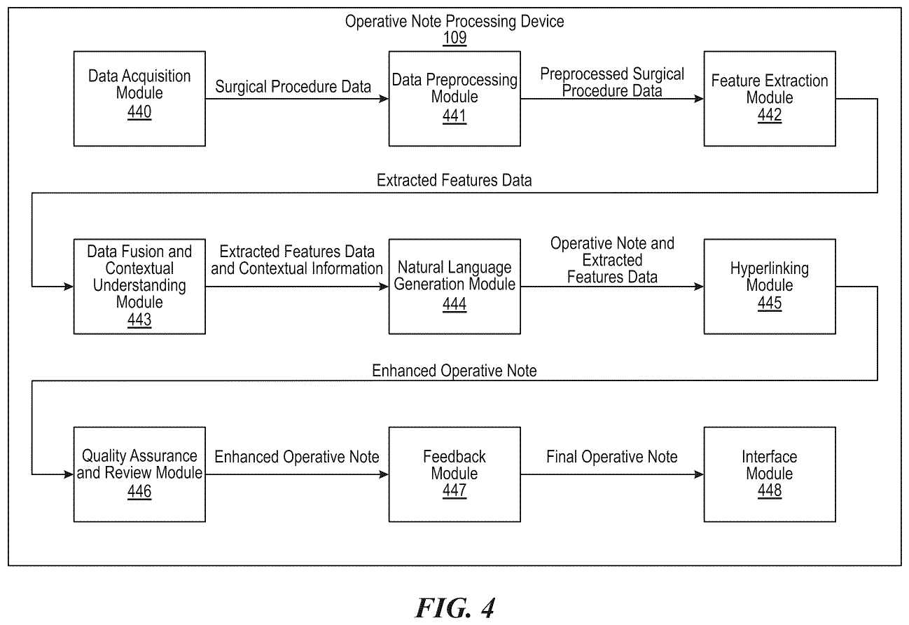

In some embodiments, the operative note processing device 109 can receive, store, and/or acquire multi-modal data of a surgical procedure carried out within the scene 108 from the sensor array 110 and/or from other sources. The multi-modal data can comprise initial image data of a patient undergoing the surgical procedure, data captured by the cameras 112 of the surgical procedure, data captured by the trackers 113 of the surgical procedure, data captured by the depth sensor 114 of the surgical procedure, data processed by the image processing device 103 (e.g., a virtual view or composite image), data processed by the registration processing device 105 (e.g., a registration of initial image data to the patient), data processed by the tracking processing device 107 (e.g., instrument positional data), and/or additional data generated before, during, and/or after the surgical procedure within the scene 108 that is relevant to the surgical procedure. Such additional data can include user inputs, user interactions, and/or the like with the system 100 such as, for example, input from a surgeon and/or technician to the system 100 to switch a view on the display device 104 to a particular vertebra (e.g., the L3 vertebra) or other structure that the surgeon is operating on. The operative note processing device 109 can utilize one or more artificial intelligence (AI) applications (e.g., machine learning (ML) models) to intelligently process the various data streams to automatically generate a detailed and accurate operative note for the surgical procedure, as described in further detail below with reference to FIGS. 4-8.

In some embodiments, functions attributed to the processing device 102, the image processing device 103, the registration processing device 105, the tracking processing device 107, and/or the data processing device 109 can be practically implemented by two or more physical devices. For example, in some embodiments a synchronization controller (not shown) controls images displayed by the projector 116 and sends synchronization signals to the cameras 112 to ensure synchronization between the cameras 112 and the projector 116 to enable fast, multi-frame, multicamera structured light scans. Additionally, such a synchronization controller can operate as a parameter server that stores hardware specific configurations such as parameters of the structured light scan, camera settings, and camera calibration data specific to the camera configuration of the sensor array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective is controlled by an input controller 106 that can update the virtual camera perspective based on user driven changes to the camera's position and rotation. The output images corresponding to the virtual camera perspective can be outputted to the display device 104. In some embodiments, the image processing device 103 can vary the perspective, the depth of field (e.g., aperture), the focus plane, and/or another parameter of the virtual camera (e.g., based on an input from the input controller) to generate different 3D output images without physically moving the sensor array 110. The display device 104 can receive output images (e.g., the synthesized 3D rendering of the scene 108) and display the output images for viewing by one or more viewers. In some embodiments, the processing device 102 receives and processes inputs from the input controller 106 and processes the captured images from the sensor array 110 to generate output images corresponding to the virtual perspective in substantially real time or near real time as perceived by a viewer of the display device 104 (e.g., at least as fast as the frame rate of the sensor array 110).

Additionally, the display device 104 can display a graphical representation on/in the image of the virtual perspective of any (i) tracked objects within the scene 108 (e.g., a surgical instrument) and/or (ii) registered or unregistered initial image data. That is, for example, the system 100 (e.g., via the display device 104) can blend augmented data into the scene 108 by overlaying and aligning information on top of “passthrough” images of the scene 108 captured by the cameras 112 and/or generated by images captured by the cameras 112. Moreover, the system 100 can create a mediated-reality experience where the scene 108 is reconstructed using light field image data of the scene 108 captured by the cameras 112, and where instruments are virtually represented in the reconstructed scene via information from the trackers 113. Additionally or alternatively, the system 100 can remove the original scene 108 and completely replace it with a registered and representative arrangement of the initial image data, thereby removing information in the scene 108 that is not pertinent to a user's task.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. In some embodiments, the system 100 can further include a separate tracking system (not shown), such an optical tracking system, for tracking the display device 104, the instrument 101, and/or other components within the scene 108. Such a tracking system can detect a position of the head-mounted display device 104 and input the position to the input controller 106. The virtual camera perspective can then be derived to correspond to the position and orientation of the head-mounted display device 104 in the same reference frame and at the calculated depth (e.g., as calculated by the depth sensor 114) such that the virtual perspective corresponds to a perspective that would be seen by a viewer wearing the head-mounted display device 104. Thus, in such embodiments the head-mounted display device 104 can provide a real time rendering of the scene 108 as it would be seen by an observer without the head-mounted display device 104. Alternatively, the input controller 106 can comprise a user-controlled control device (e.g., a mouse, pointing device, handheld controller, gesture recognition controller) that enables a viewer to manually control the virtual perspective displayed by the display device 104.

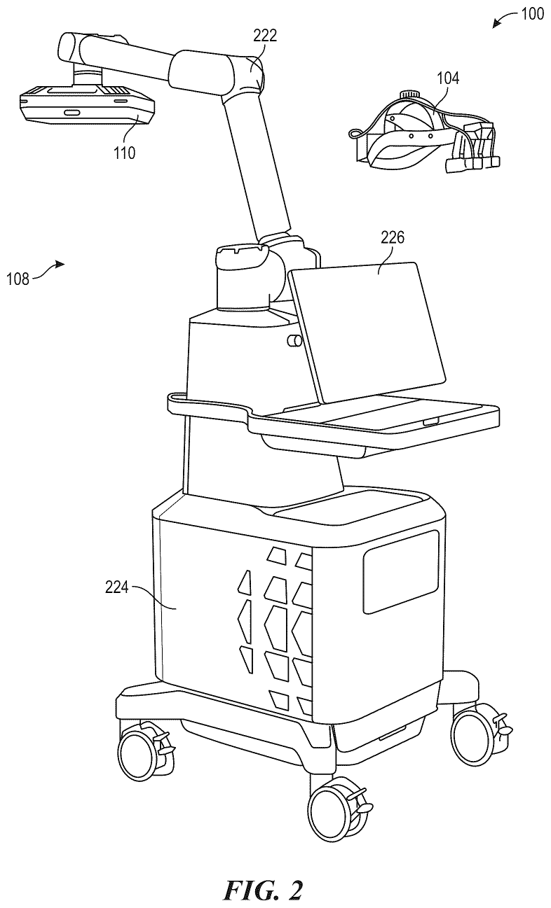

FIG. 2 is a perspective view of an environment (e.g., a surgical environment) employing the system 100 (e.g., for a surgical application) in accordance with embodiments of the present technology. In the illustrated embodiment, the sensor array 110 is positioned over the scene 108 (e.g., a surgical site) and supported/positioned via a mover 222 that is operably coupled to a workstation 224. In some embodiments, the mover 222 is manually movable to position the sensor array 110 while, in other embodiments, the mover 222 is robotically controlled in response to the input controller 106 (FIG. 1) and/or another controller. Accordingly, the mover 222 can be referred to as a robotic mover, a robotic arm, a robotically-controlled arm, and/or the like. The mover 222 allows the sensor array 110 to be precisely moved relative to the scene 108 such that the sensor array 110 is mobile relative to the scene 108.

In the illustrated embodiment, the display device 104 is a head-mounted display device (e.g., a virtual reality headset, augmented reality headset). The workstation 224 can include a computer to control various functions of the processing device 102, the display device 104, the input controller 106, the sensor array 110, and/or other components of the system 100 shown in FIG. 1. Accordingly, in some embodiments the processing device 102 and the input controller 106 are each integrated in the workstation 224. In some embodiments, the workstation 224 includes a secondary display 226 that can display a user interface for performing various configuration functions, a mirrored image of the display on the display device 104, and/or other useful visual images/indications. In other embodiments, the system 100 can include more or fewer display devices. For example, in addition to (or alternatively to) the display device 104 and the secondary display 226, the system 100 can include another display (e.g., a medical grade computer monitor) visible to the user wearing the display device 104.

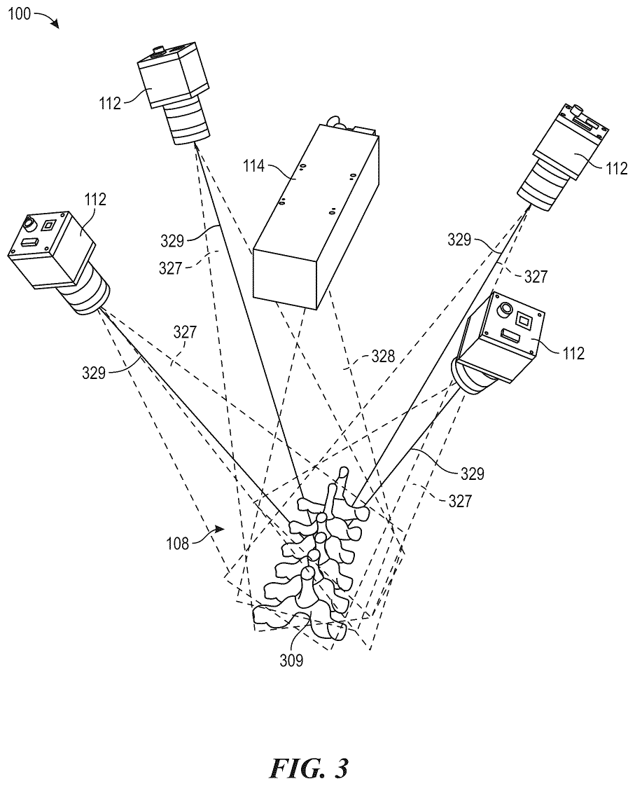

FIG. 3 is an isometric view of a portion of the system 100 illustrating four of the cameras 112 in accordance with embodiments of the present technology. Other components of the system 100 (e.g., other portions of the sensor array 110, the processing device 102, etc.) are not shown in FIG. 3 for the sake of clarity. In the illustrated embodiment, each of the cameras 112 has a field of view 327 and a focal axis 329. Likewise, the depth sensor 114 can have a field of view 328 aligned with a portion of the scene 108. The cameras 112 can be oriented such that the fields of view 327 are aligned with a portion of the scene 108 and at least partially overlap one another to together define an imaging volume. In some embodiments, some or all of the field of views 327, 328 at least partially overlap. For example, in the illustrated embodiment the fields of view 327, 328 converge toward a common measurement volume including a portion of a spine 309 of a patient (e.g., a human patient) located in/at the scene 108. In some embodiments, the cameras 112 are further oriented such that the focal axes 329 converge to a common point in the scene 108. In some aspects of the present technology, the convergence/alignment of the focal axes 329 can generally maximize disparity measurements between the cameras 112. In some embodiments, the cameras 112 and the depth sensor 114 are fixedly positioned relative to one another (e.g., rigidly mounted to a common frame) such that a relative positioning of the cameras 112 and the depth sensor 114 relative to one another is known and/or can be readily determined via a calibration process. In other embodiments, the system 100 can include a different number of the cameras 112 and/or the cameras 112 can be positioned differently relative to another.

Referring to FIGS. 1-3 together, in some aspects of the present technology the system 100 can generate a digitized view of the scene 108 that provides a user (e.g., a surgeon) with increased “volumetric intelligence” of the scene 108. For example, the digitized scene 108 can be presented to the user from the perspective, orientation, and/or viewpoint of their eyes such that they effectively view the scene 108 as though they were not viewing the digitized image (e.g., as though they were not wearing the head-mounted display 104). However, the digitized scene 108 permits the user to digitally rotate, zoom, crop, or otherwise enhance their view to, for example, facilitate a surgical workflow. Likewise, initial image data, such as CT scans and/or MRI data, can be registered to and overlaid over the image of the scene 108 to allow a surgeon to view these data sets together. Such a fused view can allow the surgeon to visualize aspects of a surgical site that may be obscured in the physical scene 108-such as regions of bone and/or tissue that have not been surgically exposed.

### II. Selected Embodiments of Systems and Methods for Automatically Generating a Surgical Operative Note

Referring to FIGS. 1-3, the system 100 can capture and/or generate robust, multi-modal data of a surgical procedure such as image data, instrument tracking data, registration data, depth data, user interactions with the system, user inputs to the system, and/or the like in real time or near real time over the course of a surgical procedure. The data processing device 109 can process some or all of the collected data, and optionally data from sources other than sensor array 110, to automatically generate an accurate surgical operative note describing the surgical procedure. A surgical operative note is a medical document that provides a detailed record of the surgical procedure performed including, for example, the date and time of the surgery, the name of the surgeon and surgical team members, the type of procedure performed, a step-by-step description of the surgical technique, etc. The operative note plays an integral role in: (i) patient care by ensuring that all healthcare team members have access to accurate and up-to-date information about the surgical procedure, (ii) fulfilling legal and regulatory requirements, (iii) the billing process for the surgical procedure, (iv) educating and training medical students, residents, and other healthcare professionals, (v) research and quality improvement initiatives, and (vi) follow up care and monitoring for the patient. An operative note must be accurate and detailed to fulfill its myriad of roles.

Currently, writing or developing an accurate surgical operative note manually involves various challenges and hurdles that healthcare providers need to overcome. For example, surgical procedures can be complex and dynamic-with multiple steps, variations, and unexpected findings. Keeping track of all intraoperative events and accurately documenting them in real time can be challenging, especially in high-stress and time-sensitive situations. Likewise, surgeons and surgical team members often face time constraints during procedures, limiting the time available for documenting intraoperative details. Balancing the need for thorough documentation with the need to focus on patient care and surgical tasks can be difficult. Additionally, the interpretation of intraoperative findings and events can be subjective, leading to variability in how different healthcare providers document and describe the same procedure. Achieving consistency and standardization in manual operative note documentation across surgical teams and specialties can be challenging. Healthcare providers may also receive limited training and education on operative note documentation practices, leading to inconsistencies, inaccuracies, or omissions in documentation. Furthermore, electronic health record (EHR) systems used for operative note documentation may have usability issues, such as cumbersome interfaces, inefficient workflows, and/or lack of integration with surgical workflow processes that can hinder the efficient and accurate documentation of operative notes. Healthcare providers also often face documentation burden due to the need to document a wide range of clinical information, including preoperative assessments, intraoperative details, and postoperative care. The documentation burden can lead to fatigue, errors, and/or incomplete documentation in an operative note. Additionally, surgical procedures often involve collaboration among multiple healthcare providers, including surgeons, anesthesiologists, nurses, and other surgical team members. Coordinating and integrating contributions from different team members into the operative note while maintaining accuracy and consistency can be challenging.

Existing documentation tools and systems have severe limitations in capturing and representing intraoperative information effectively. Even current state-of-the-art technologies, such as voice recognition software or mobile documentation tools, do not solve the problem of time constraints, documentation burden, and interdisciplinary collaboration.

FIG. 4 is a block diagram of the operative note processing device 109 of FIG. 1 in accordance with embodiments of the present technology. In general, the operative note processing device 109 is configured to automatically generate a surgical operative note by leveraging multi-modal data captured and/or generated by the system 100 of FIG. 1 (e.g., the sensor array 110) and/or from data sources other than the system 100 to produce a detailed and accurate operative note of a surgical procedure. In the illustrated embodiment, the operative processing device 109 includes a data acquisition module 440, a data preprocessing module 441, a feature/object extraction module 442, a data fusion and contextual understanding module 443, a natural language generation module 444, a hyperlinking module 445, a quality assurance and review module 446, a feedback module 447, and an interface module 448 (collectively modules 440-448). The modules 440-448 cooperate to perform a method of automatically generating an operative note.

The data acquisition module 440 can acquire, record, and/or store many forms of data related to a surgical procedure carried out on a patient, such as a spinal surgical procedure, a general surgical procedure, an orthopedic surgical procedure, a neurosurgical procedure, a laparoscopic procedure, etc. For example, the data acquisition module 440 can receive intraoperative video data, tracking data, depth data, and/or the like from one or more video recording devices, depth cameras, endoscopes, and/or the like. For example, referring also to FIG. 1, the data acquisition module 440 can receive video data from the cameras 112 (e.g., RGB video data), video data from the trackers 113 (e.g., infrared video data), depth data from the depth sensor 114, etc. In some embodiments, the data acquisition module 440 receives data directly from the sensor array 110 while, in other embodiments, the data acquisition module 440 receives data processed by the image processing device 103, the registration processing device 105, and/or the tracking processing device 107. For example, the data acquisition module 440 can receive raw video data from the cameras 112 and also a synthetic video stream of the surgical procedure generated by the image processing device 103 based on multiple video streams from the cameras 112.

In addition to intraoperative data captured by the sensor array 110 and/or other intraoperative instruments (e.g., an endoscope), the data acquisition module 440 can receive other types of data such as (i) initial image data of the patient (e.g., computerized tomography (CT) images, magnetic resonance imaging (MRI) images and/or the like acquired preoperatively, during, or shortly before the surgical procedure), (ii) surgical navigation and planning data, (iii) log data, (iv) electronic health records (EHRs) of the patient, (v) surgical instrument data (e.g., kind, size, type), (vi) user inputs to and/or interactions with the system 100 (e.g., a user input to change a view on the display device 104) and/or (vii) the like. The data acquired by the data acquisition module 440, whether video data, preoperative imaging data, log data, etc., can be referred to as “surgical procedure data.” In some embodiments, the surgical procedure data is stored in a digital format for further processing by the operative note processing device 109.

The data preprocessing module 441 can receive the surgical procedure data from the data acquisition module 440 and preprocess the surgical procedure data to enhance its quality, remove noise, and/or integrate different data modalities. For example, referring also to FIG. 1, the data preprocessing module 441 can receive raw RGB video data captured by the cameras 112 and infrared video data captured by the trackers 113 and process the RGB and infrared video data to enhance their quality while also integrating the two streams of video data captured by different camera modalities. The preprocessing module can also convert image (e.g., pre-operative images) and text data (e.g., log data) into a form compatible with the video data. In some embodiments, the data preprocessing module 441 can utilize one or more artificial intelligence (AI) applications to process the surgical procedure data. For example, an AI application can process video data to detect changes in pixelation in the video data that indicate an obstruction in the video data at a particular time. The data preprocessing module 441 can then filter out such obstructed video frames that provide little or no information about the surgical procedure. Accordingly, the data preprocessing module can output preprocessed surgical procedure data.

The feature extraction module 442 can analyze the preprocessed surgical procedure data to extract (e.g., recognize) relevant features, including surgical actions, anatomical landmarks, instruments and objects, instrument and object movements, and intraoperative events. Specifically, the feature extraction and tracking module 442 can utilize computer vision techniques such as object detection, motion tracking, and/or image segmentation to identify and extract these features from the processed surgical procedure video data. In some embodiments, the feature extraction module 442 utilizes an artificial intelligence (AI) application that receives as inputs the preprocessed surgical procedure data and that outputs the relevant features. The AI application can be a two-stage temporal convolutional model. Such models function by first using an image or “clip” model to generate an embedding from each video frame or short sequence of frames. The embeddings are then stacked temporally to create an embedded representation of the full video. This sequence of embeddings is then fed into a “sequencer” model which is usually a multistage convolutional model, such as MS-TCN, transformer based architectures, or even language models like BERT. The sequencer model then provides temporal context to the embeddings as well as providing temporal smoothing to generate the final computer vision workflow predictions (e.g., extracted features).

In some embodiments, such a two-stage temporal convolutional model can be of the type described in (i) “Surgical workflow recognition with temporal convolution and transformer for action segmentation,” published in the International of Computer Assisted Radiology and Surgery, by B. Zhang, B. Goel, M. H. Sarhan, V. K. Goel, R. Abukhalil, B. Kalesan, N. Stottler, and S. Petculescu, 2023; 18(4):785-794. doi:10.1007/s11548-022-02811-z, and available at https://pubmed.ncbi.nlm.nih.gov/36542253/ and/or (ii) “MS-TCN++: Multi-Stage Temporal Convolutional Network for Action Segmentation,” published in IEEE Transactions on Pattern Analysis and Machine Intelligence, by S. Li, Y. A. Farha, Y. Liu, M.-M. Cheng, and J. Gall, vol. 45, no. 6, pp. 6647-6658, 1 Jun. 2023, doi:10.1109/TPAMI.2020.3021756, and available at https://ieeexplore.ieee.org/document/9186840, each of which is hereby incorporated by reference in its entirety.

Features that can be extracted from the video data can include (i) surgical actions such as blunt dissection, deep dissection, incision, closure, laminotomy, etc., (ii) anatomical landmarks such spinous processes, inter-spinous ligaments, lamina, pars and facets, etc., (iii) instruments, objects, hardware, tools, implants, etc., (iv) instrument and object movements such as pedicle screw entry, cutting instrument usage, retractor usage, etc., and/or (v) intraoperative events such as incision, dissection, closure, etc. In some embodiments, the feature extraction module 442 utilizes preprocessed tracking data from the trackers 113 (FIG. 1) to recognize instrument movements, and can compare the integrated video data from the cameras 112 to determine corresponding surgical actions and intraoperative events. For example, if a cutting instrument is recognized as approaching the anatomy of the patient in the tracking data from the trackers 113, the feature extraction module 442 can analyze the corresponding video data from the cameras 112 to determine a corresponding surgical action (e.g., dissection, laminotomy) and/or intraoperative event (e.g., incision, dissection). In some embodiments, the feature extraction module 442 can segment video data into relevant segments corresponding to different phases of the surgical procedure. For example, for an open surgical procedure, the data preprocessing module 441 can segment the surgical procedure into an “incision” phase, a “dissection” phase, and a “closure” phase.

The outputs of the feature extraction module 442 can be portions of the surgical procedure data that correspond to an identified feature/object, such as video frames (e.g., video snippets, video segments), preoperative images, surgical navigation data, etc. For example, when the feature extraction module 442 identifies a dissection in the surgical procedure data, the feature extraction module 442 can output an image of the dissection from a single video frame, and/or can output a video segment showing the incision being made. Likewise, where the feature extraction module 442 identifies a laminotomy in the surgical procedure data, the feature extraction module 442 can output an image of the completed laminotomy, a video segment showing the laminotomy being carried out, a preoperative image of the vertebra before the laminotomy, data about an instrument identified as used to carry out the laminotomy, etc.

In some embodiments, the feature extraction module 442 can extract and detect features according to a hierarchical framework and/or in accordance with a standardized manner of describing a surgical procedure. For example, the extracted features can be annotated/labeled in accordance with the “steps” and “tasks” framework developed by the Society of American Gastrointestinal and Endoscopic Surgeons (SAGES). More particularly, features can be labeled with a phase, step, task, and/or action. The phase can be a generic label representing the highest-level temporal component of the surgical procedure, such as: surgical access (e.g., spinal exposure), execution of surgical objectives (e.g., surgical steps carried out the spine), and closure (e.g., surgical closure of the access to the spine). The step can be a procedure-specific label representing a specific segment of the surgical procedure carried out to accomplish a clinically meaningful goal, without which the procedure cannot be completed (e.g., resection of a vertebra, implantation of a pedicle screw, placement of a spinal implant). Steps need not be performed in a specific order, can be interrupted, and do not have to be unique to the particular surgical procedure (e.g., the same step can be present across similar surgical procedures). The task can be a sub-component of a step carried out to accomplish the goal of the step (e.g., trajectory determination for a pedicle screw, insertion of the pedicle screw). Multiple tasks often must be completed to carry out a step. The action can be a primitive component of a task (e.g., align, insert) and multiple actions are often required to complete a task. The action can be represented as a verb. Similarly, the feature extraction module 442 can label/describe features according to different frameworks and/or with different descriptors. For example, tasks can be associated with a dominate action type (e.g., suturing, dissection, cannulation) targeted at a particular anatomy or surgical object (e.g., a particular vertebra).

The data fusion and contextual understanding module 443 can receive the extracted features as a data stream from the feature extraction module 442 and integrate the extracted features from multiple data modalities to provide context and temporal understanding of the surgical procedure. For example, the data fusion and contextual understanding module 443 can group the same feature recognized in different modalities of the surgical procedure data together to provide a temporal understanding of the surgical procedure (e.g., by syncing times, audio recordings, textual event logs, etc.). As one example, the data fusion and contextual understanding module 443 can group together an extracted video segment of a laminotomy captured by one or more of the cameras 112 (FIG. 1), extracted depth information of the vertebra before, during, and/or after the laminotomy from the depth sensor 114 (FIG. 1), extracted surgical instrument data from the trackers 113 (FIG. 1; e.g., the type of instrument used to carry out the laminotomy, its position/trajectory during the laminotomy, etc.), a preoperative image of the vertebra before the laminotomy, surgical navigation data during the laminotomy, etc.

The data fusion and contextual understanding module 443 can also provide additional contextual information/data based on the extracted features to provide context to the surgical procedure. For example, the data fusion and contextual understanding module 443 can utilize an artificial intelligence (AI) application (e.g., a generative AI application, a generative AI model, a large language model (LLM), and/or the like) that receives as inputs one or more of the extracted features and/or additional surgical procedure data such as EHRs (e.g., including patient demographics, surgical indications, and preoperative assessments), preoperative images, audio data recorded during the surgical procedure, user inputs to and/or interactions with the system 100, a textual event log of the surgical procedure (e.g., a record of every action taken during the surgical procedure and logged into a database) and/or the like and that outputs contextual information about the surgical procedure. For example, EHR data including patent symptoms and preoperative images can inform the AI application about what surgical procedure would most likely be adopted for the particular surgical procedure carried out. As a more specific example, for a spinal surgical procedure, a preoperative CT image of the patient that reveals past L3-L4 fusion along with the knowledge of symptoms such as lumbar pain radiating bilaterally can inform the model that among the likely surgical procedures performed could be Revision L3-L5 Posterior Spinal Instrumented Fusion (Revision PSIF). Such contextual information can be added to the various extracted features—for example, that a video snippet of an incision and retraction in the patient is to access the L3-L5 vertebrae for fusion. Similarly, the data fusion and contextual understanding module 443 can utilize data of user interactions with and/or user inputs to the system 100 to determine corresponding surgical actions and/or intraoperative events. For example, a user interaction (e.g., via a surgeon or technician) with the system 100 to switch a view on the display device 104 to a particular vertebra (e.g., the L3 vertebra) or other structure that the surgeon is operating on can be used to provide contextual information for the various extracted features—for example, that a video snippet of a surgical action is of the particular structure (e.g., L3 vertebra).

The natural language generation module 444 can receive the fusion of extracted features and contextual information from the data fusion and contextual understanding module 443 and utilize an AI application to convert the extracted features and contextual information into natural language descriptions. In some embodiments, the AI application is a natural language processing (NLP) algorithm that utilizes machine learning to convert video and other data to natural language text data. The outputs of the natural language generation module 444 is a structured and coherent operative note of the surgical procedure that summarizes the surgical procedure, including the type of surgery performed, specific surgical techniques used, intraoperative findings, and/or postoperative care instructions.

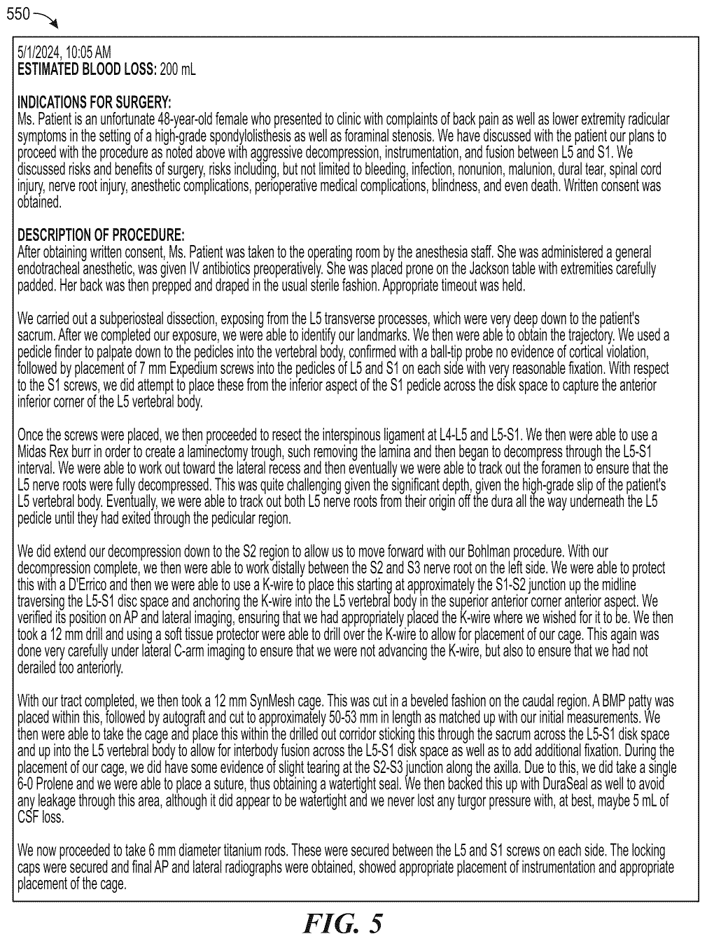

FIG. 5, for example, illustrates an example of an operative note 550 generated by the natural language generation module 444 and that can be displayed on a display screen, computing device, user interface, and/or the like in accordance with embodiments of the present technology. In the illustrated embodiment, the operative note 550 includes a text-based summary of a spinal surgical procedure carried out on a 48-year-old female patient based on surgical procedure data acquired, processed, and analyzed according to the modules 440-443. The text in the operative note 550 can be generated in natural language from the perspective of the surgical team carrying out the surgical procedure (and/or from another desired perspective) and can be generated automatically—obviating the need for the surgeon and/or other surgical team members to recall and manually document the surgical procedure after its conclusion.

Referring again to FIG. 4, the AI applications utilized by the data fusion and contextual understanding module 443 and/or the natural language generation module 444 can be, for example, a foundational model (e.g., one of OpenAI's GPT models) that is pre-trained on domain specific surgical procedure data. That is, the foundational model can be trained to the specific domain of surgical procedures and/or more specifically to a specific type of surgical procedure, such as spinal surgical procedures.

The hyperlinking module 445 can receive the operative note from the natural language generation module and the extracted features (e.g., from the feature extraction module 442 and/or the data fusion and contextual understanding module 443) and embed hyperlinks and/or other indicia into the operative note that link textual descriptions in the operative note to corresponding extracted features in the surgical procedure data. For example, the hyperlinking module 445 can embed a hyperlink in the operative note that links to a video segment and/or image from the appropriate, corresponding part of the surgical procedure. User selection of the hyperlink can cause the video segment (or image or other data modality) to display to a viewer (e.g., a surgeon, a doctor, a medical student, a medical resident) when the operative note is viewed on a computing device. The hyperlinks can enable the viewer to quickly access and view detailed video and/or other surgical procedure data corresponding to a textual description in the operative note. The operative note with embedded hyperlinks can be referred to as an “enhanced operative note.”

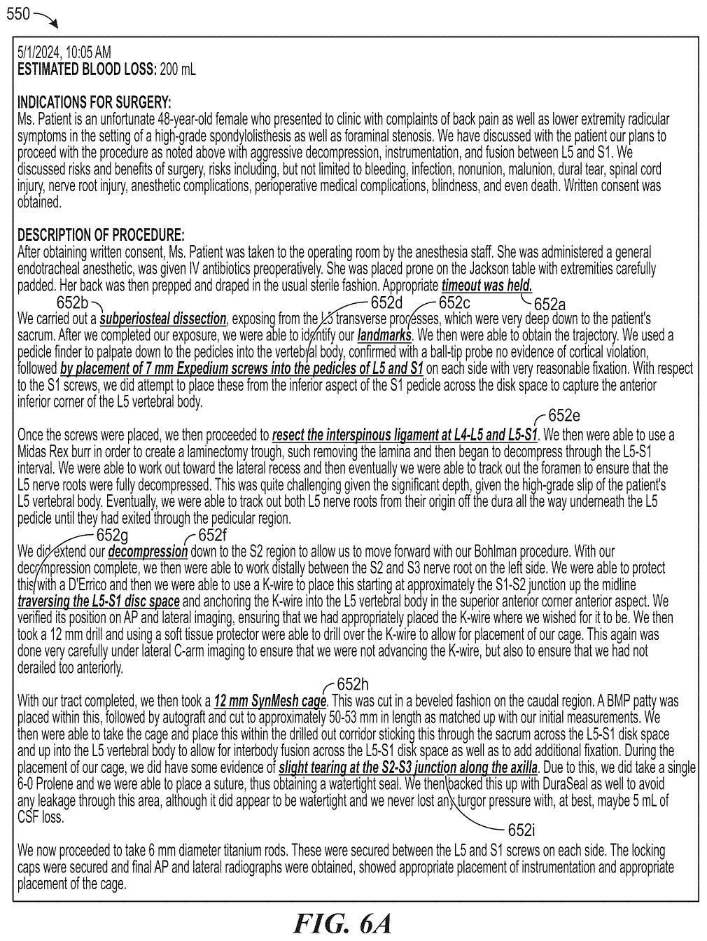

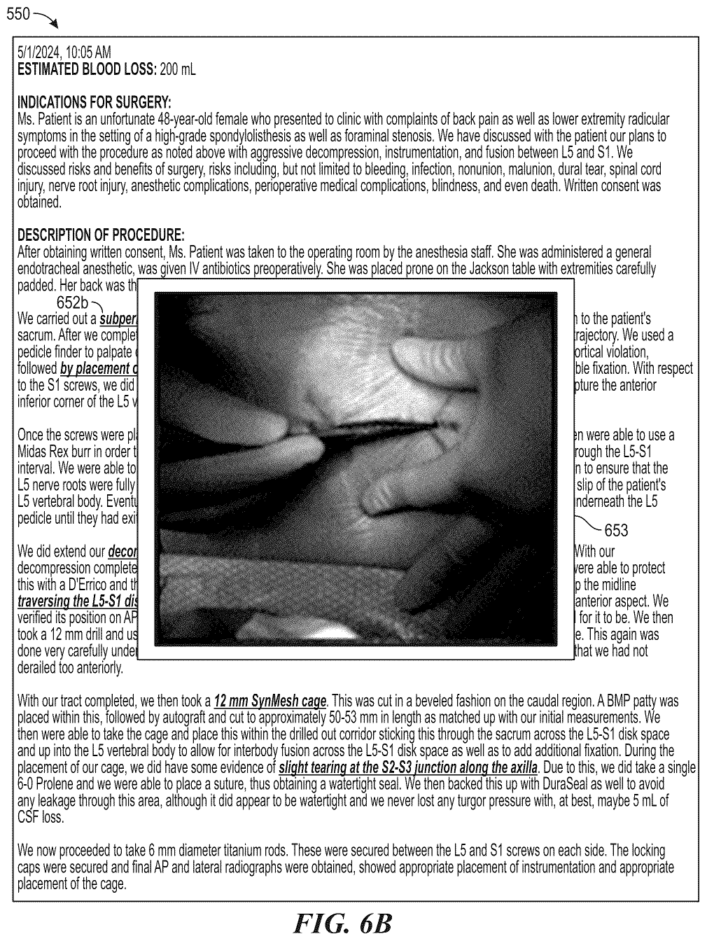

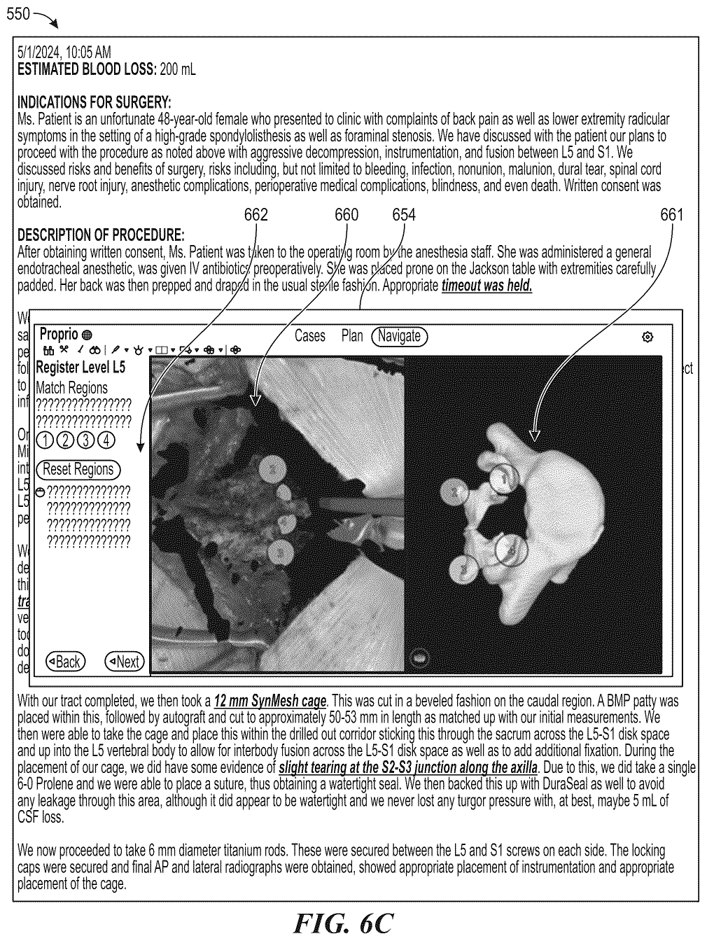

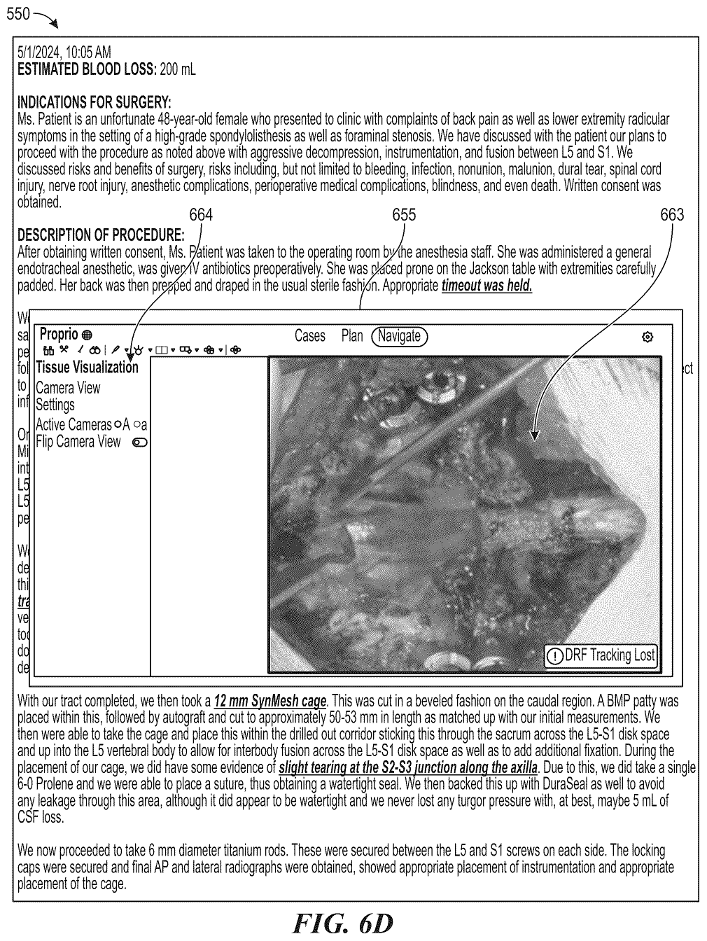

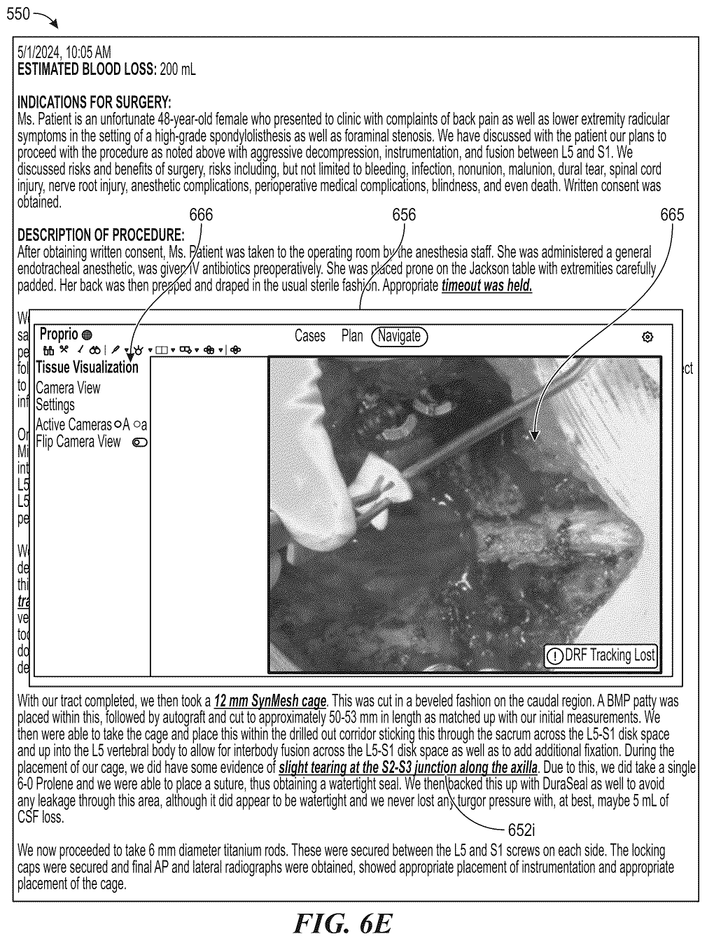

FIG. 6A, for example, illustrates the operative note 550 of FIG. 5 enhanced with hyperlinks 652 (individually identified as first through ninth hyperlinks 652a-i, respectively) embedded therein via the hyperlinking module 445 in accordance with embodiments of the present technology. In the illustrated embodiment, (i) the first hyperlink 652a can provide a link from the generated text “timeout was held” to a corresponding video segment, image, and/or other data modality related to a surgical timeout during the surgical procedure, (ii) the second hyperlink 652b can provide a link from the generated text “subperiosteal dissection” to a corresponding video segment, image, and/or other data modality related to a dissection (e.g., an initial dissection, a subperiosteal dissection) during the surgical procedure, (iii) the third hyperlink 652c can provide a link from the generated text “landmarks” to a corresponding video segment, image, and/or other data modality related to one or more landmarks (e.g., landmarks used to carry out a registration by the system 100 of FIG. 1) identified during the surgical procedure, (iv) the fourth hyperlink 652d can provide a link from the generated text “by placement of 7 mm Expedium screws into the pedicles of L5 and S1” to a corresponding video segment, image, and/or other data modality related to placement of screws in the lumbar L5 vertebra and sacral S1 vertebra during the surgical procedure, (v) the fifth hyperlink 652e can provide a link from the generated text “resect the interspinous ligament at L4-L5 and L5-S1” to a corresponding video segment, image, and/or other data modality related to resection of the interspinous ligament at the L4-L5 vertebrae and at the L5-S1 vertebrae during the surgical procedure, (vi) the sixth hyperlink 652f can provide a link from the generated text “decompression” to a corresponding video segment, image, and/or other data modality related to a decompression during the surgical procedure, (vii) the seventh hyperlink 652g can provide a link from the generated text “traversing the L5-S1 disc space” to a corresponding video segment, image, and/or other data modality related to a traversal of the L5-S1 disc space with a K-wire during the surgical procedure, (viii) the eighth hyperlink 652h can provide a link from the generated text “12 mm SynMesh cage” to a corresponding video segment, image, and/or other data modality related to the placement, implantation, and/or selection of a cage (e.g., a 12 mm SynMesh cage) during the surgical procedure, and (ix) the ninth hyperlink 652i can provide a link from the generated text “slight tearing at the S2-S3 junction along the axilla” to a corresponding video segment, image, and/or other data modality related to an identified dural tear (e.g., at the S2-S3 junction along the axilla) during the surgical procedure. Depending on the specific surgical procedure and collected surgical procedure data, the operative note 550 can have more, fewer, and/or different ones of the hyperlinks 652, and/or the text included in the hyperlink may vary (e.g., hyperlinking the text “resect” instead of “resect the interspinous ligament at L4-L5 and L5-S1” for the fifth hyperlink 652e).

More specifically, FIGS. 6B-6E illustrate various views of the enhanced operative note 550 of FIG. 6A after a user selection of different ones of the hyperlinks 652 in accordance with embodiments of the present technology. Referring to FIG. 6B, selection of the second hyperlink 652b can cause the display of an image or video segment 653 showing subperiosteal dissection during the surgical procedure. In some embodiments, the image or video segment 653 comprises raw video data captured by, for example, one or more of the cameras 112 of FIG. 1.

Referring to FIG. 6C, selection of the third hyperlink 652c (FIG. 6A) can cause the display of an image or video segment 654 showing the identification of the landmarks during the surgical procedure. In some embodiments, the image or video segment 654 is a navigated display or navigated display interface from the surgical procedure such as, for example, any of those described in U.S. patent application Ser. No. 17/864,065, filed Jul. 13, 2022, and titled “METHODS AND SYSTEMS FOR DISPLAYING PREOPERATIVE AND INTRAOPERATIVE IMAGE DATA OF A SCENE,” which is incorporated herein by reference in its entirety. Such a navigated display can be generated by the system 100 (FIG. 1). For example, the image or video segment 654 can include a display of an intraoperative image 660 (e.g., including a surgically-exposed vertebra), a corresponding preoperative or initial image 661 (e.g., a CT image of the corresponding vertebra), and a registration interface 662. The registration interface 662 can be used during the surgical procedure to guide registration of the initial image 661 to the intraoperative image 660. Accordingly, the image or video segment 654 can show the registration of the identified landmark (e.g., the lumbar L5 vertebra) to the initial image 660 during the surgical procedure.

Referring to FIG. 6D, selection of the sixth hyperlink 652f (FIG. 6A) can cause the display of an image or video segment 655 showing the decompression step during the surgical procedure. The image or video segment 655 can, similar to the image or video segment 654, comprise a navigated display or navigated display interface including a display of an intraoperative image 663 (e.g., including a surgically-exposed vertebra) and a visualization interface 664. The visualization interface 664 can allow for user input to adjust the intraoperative image 663 during the surgical procedure.

Referring to FIG. 6E, selection of the ninth hyperlink 652i can cause the display of an image or video segment 656 showing the identification and/or treatment of the dural tearing at the S2-S3 junction along the axilla during the surgical procedure. The image or video segment 656 can, similar to the image or video segment 655, comprise a navigated display or navigated display interface including a display of an intraoperative image 665 (e.g., including a surgically-exposed vertebra) and a visualization interface 666.

Referring to FIGS. 6B-6E, the image or video segments 653-656 can be displayed on the display screen or other device used to display the operative note 550 (and/or on a separate display screen) as (i) a pop-up on the display screen over, adjacent, or otherwise near the operative note 550, (ii) as a separate window (e.g., without corresponding display of the operative note 550), and/or (iii) in other manners.

Referring again to FIG. 4, the quality assurance and review module 446 can receive the enhanced operative note generated by the video hyperlinking module and validate the accuracy and completeness of the operative note through automated checks. Such automated checks can determine an objective score of descriptive accuracy of the operative note. In some embodiments, the quality assurance and review module 446 can identify and correct any discrepancies or errors in the generated operative note to ensure its reliability and compliance with medical standards and documentation guidelines.

The feedback module 447 can receive the enhanced operative note from the quality assurance and review module 446 and solicit and/or receive feedback from a viewer of the operative note. For example, the feedback module 447 can present the enhanced operative note on a display device and solicit user feedback to verify the accuracy and completeness of the operative note.

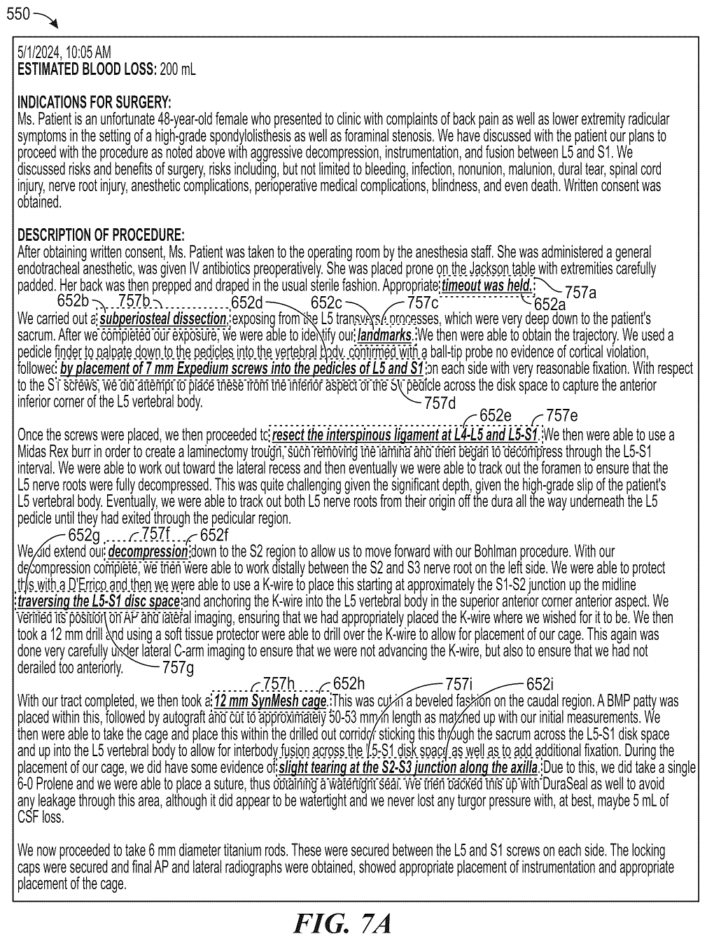

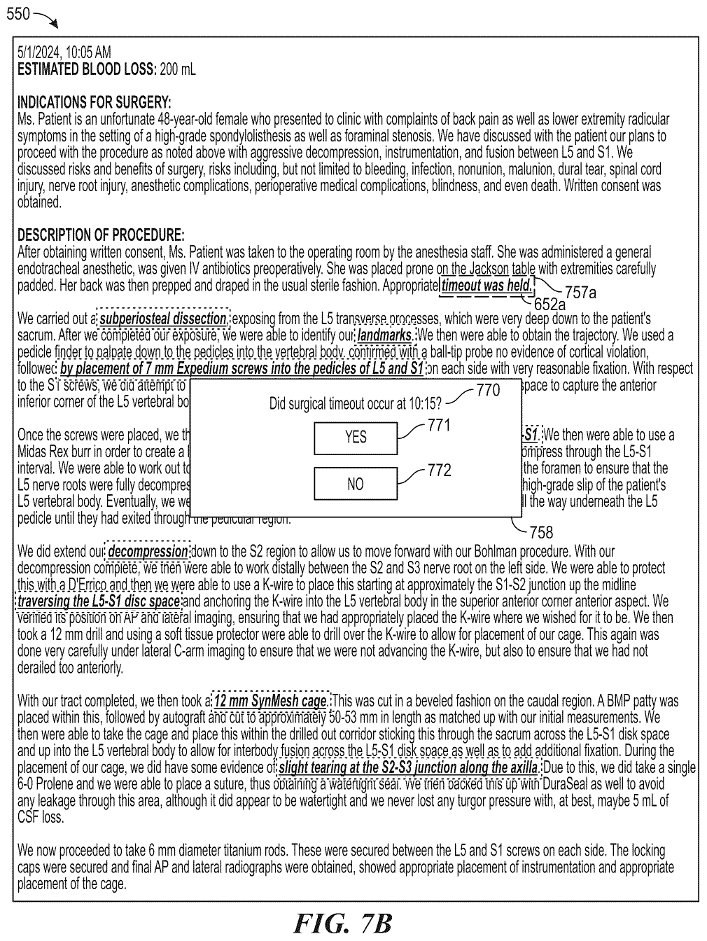

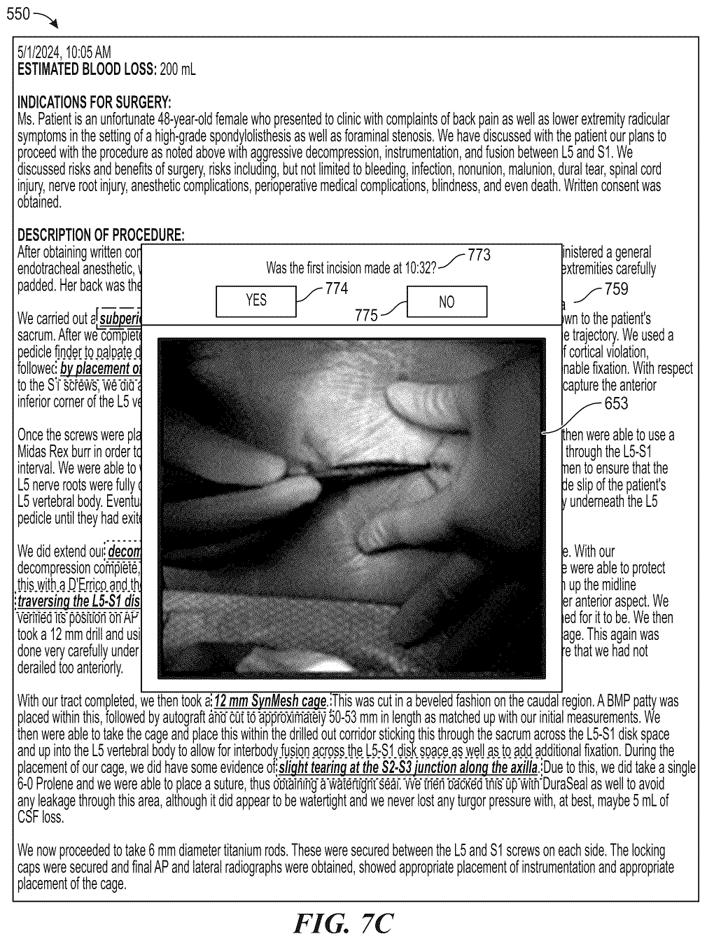

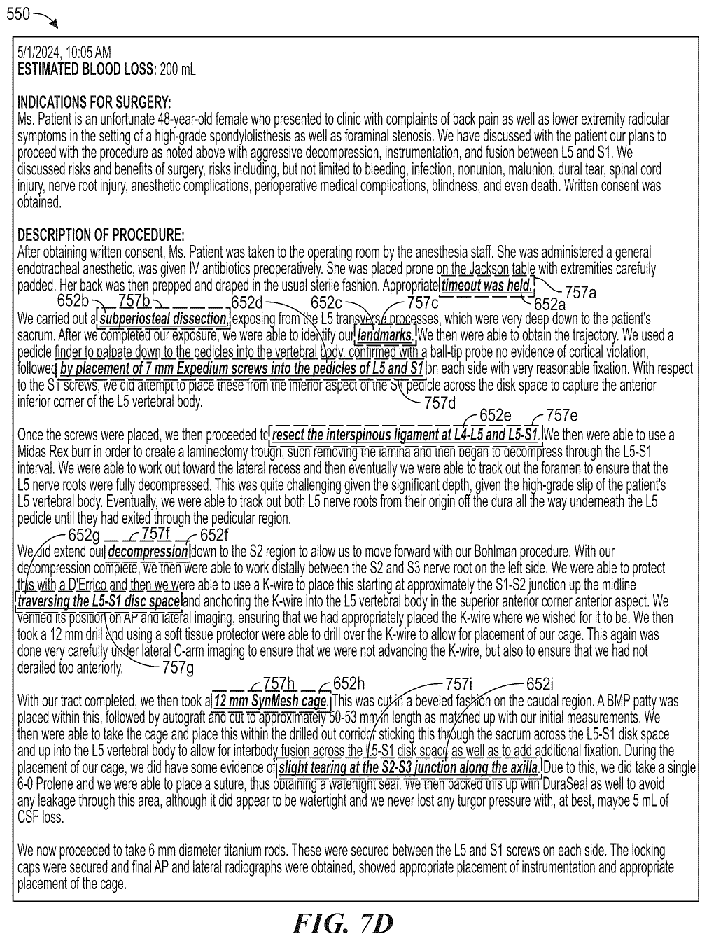

More specifically, for example, FIGS. 7A-7D illustrate the display of the enhanced operative note 650 of FIG. 6A with the solicitation of user feedback in accordance with embodiments of the present technology. Referring to FIG. 7A, the feedback module 447 (FIG. 4) can generate visual indicators (individually identified as first through ninth indicators 757a-i, respectively) for portions of the operative note 550 for which feedback is needed or desired. In the illustrated embodiment, the indicators 757 are boxes around corresponding ones of the hyperlinks 652. In other embodiments, the feedback module 447 (FIG. 4) may not solicit feedback for each of the hyperlinks 652 such that the some or all of the hyperlinks are not identified by a corresponding one of the indicators 757. In some embodiments, prior to user feedback, the indicators 757 each have a first representation indicating that feedback is needed or desired for the corresponding portion of the operative note 550. For example, in the illustrated embodiment the indicators 757 each have a first color (e.g., red) and a first style (e.g., dashed lines).

A user can individually select one the hyperlinks 652 and/or the corresponding indicator 757 to provide feedback. For example, referring to FIG. 7B, user selection of the first hyperlink 652a and/or the first indicator 757a can cause the display of a feedback window 758 soliciting feedback from a user (e.g., a surgeon or other surgical team member) to confirm the accuracy of the corresponding portion of the operative note (e.g., “timeout was held”). The feedback window 758 can include a prompt 770 prompting the user for specific feedback regarding the portion of the operative note 550 (e.g., “Did surgical timeout occur at 10:15?”), a validate button 771 (e.g., “YES”), and an invalidate button 772 (e.g., “NO”). The user can confirm or deny the accuracy of the portion of the operative note 550 by selecting either the validate button 771 or the invalidate button 772. If the user selects the validate button 771, the feedback module 447 (FIG. 4) can automatically change the first indicator 757a to have a second representation indicating that feedback is no longer needed for that portion of the operative note 550. For example, in the illustrated embodiment the first indicator 757a now has a second color (e.g., green) and a second style (e.g., solid lines). In some embodiments, if the user selects the invalidate button 772, the feedback module 447 (FIG. 4) can permit the user to edit and/or modify the operative note 550 to describe the surgical procedure more accurately and/or more completely.

In some embodiments, a feedback window presented by the feedback module 447 (FIG. 4) can include a display of an image or video segment from the surgical procedure corresponding to the portion of the operative note 550 to help the user in determining the accuracy of the portion of the operative note 550. Referring to FIG. 7C, for example, user selection of the second hyperlink 652b and/or the second indicator 757b can cause the display of a feedback window 759 soliciting feedback from the user to confirm the accuracy of the corresponding portion of the operative note (e.g., “subperiosteal dissection”). Similar to the feedback window 758 of FIG. 7B, the feedback window 759 can include a prompt 773 prompting the user for specific feedback regarding the portion of the operative note 550 (e.g., “Was the first incision made at 10:32?”), a validate button 774 (e.g., “YES”), and an invalidate button 775 (e.g., “NO”). In the illustrated embodiment, the feedback window 759 can further include the image or video segment 653 showing the dissection of subperiosteal dissection during the surgical procedure, as described in detail above with reference to FIG. 6B. The image or video segment can help the user in determining the accuracy of the portion of the operative note 550 by providing them a specific visual cue or reminder of what actually happened during the corresponding portion of the surgical procedure. Again, if the user selects the validate button 774, the feedback module 447 (FIG. 4) can automatically change the second indicator 757b to have a second representation (e.g., green and solid lines) indicating that feedback is no longer needed for that portion of the operative note 550.

Referring to FIG. 7D, the feedback module 447 (FIG. 4) can present a feedback window and, in some instances, a corresponding image or video segment as the user selects each of the hyperlinks 652 and/or indicators 757. After the user has provided feedback for each of the hyperlinks 652 and/or indicators 757, the indicators 757 can each have the second representation (e.g., green and solid lines) indicating that feedback is complete. In general, the feedback windows (e.g., the feedback windows 758 and 759 of FIGS. 7B and 7C) can be displayed on the display screen or another device used to display the operative note 550 (and/or on a separate display screen) as (i) a pop-up on the display screen over, adjacent, or otherwise near the operative note 550, (ii) as a separate window (e.g., without corresponding display of the operative note 550), and/or (iii) in other manners.

Referring again to FIG. 4, after all feedback is solicited and the operative note is tuned/updated as needed, the feedback module 447 can output a final operative note. In some embodiments, the interface module 448 receives the final operative note and is configured to interface with one or more clinical health care systems, financial systems, and/or the like (e.g., third party systems and/or applications). For example, the feedback module 447 can store the final operative note—and the final operative notes generated for multiple surgical procedures—and can be configured to interface with the one or more clinical health care systems, financial systems, and/or the like to provide a given final operative note upon request. In some embodiments, the interface module 448 interfaces and/or comprises an application programming interface (API) that can receive API calls/requests from the one or more clinical health care systems, financial systems, and/or the like to provide a given final operative note. For example, financial systems such as revenue cycle management (RCM) systems, billing systems, insurance systems, and/or the like may request an operative note in order to verify the medical necessity of the surgical procedure, ensure appropriate coding, calculate the reimbursement amount based on established fee schedules or reimbursement rates, etc. Likewise, clinical health care systems such as hospital systems, medical school systems, and/or the like may request an operative note to inform ongoing postoperative care for the patient, provide teaching and learning opportunities, etc.

In some embodiments, automatic updates (e.g., changes, modifications, corrections) made to the operative note via the quality assurance and review module 446 and/or manual (e.g., user-driven) updates made to the operative note via the feedback module 447 can be used to train and the AI applications utilized by the data fusion and contextual understanding module 443 and/or the natural language generation module 444. For example, such updates can be used as reinforcement learning training for the AI applications. More specifically, a reward function can be defined that accurately reflects the quality of the operative note (e.g., with fewer updates implemented by the quality assurance and review module 446 and/or the feedback module 447 indicating a higher quality). Reinforcement learning algorithms can be used to update model parameters of the AI applications to maximize the expected rewards over time. The models can be fine-tuned iteratively based on rewards received and evaluation of the quality of the operative note generated.

Referring to FIGS. 1 and 4, the operative note processing device 109 can be installed in the system 100 and configured to run/operate within the system 100 without a connection to the internet, an external cloud application, and/or the like. That is, the operative note processing device 109 can be positioned local to (e.g., integrated within) the system 100. In other embodiments, the operative note processing device 109 can be deployed in a cloud computing environment and connected to the system 100 through an internet connection, such as a secure internet connection with sufficient bandwidth. That is, the operative note processing device 109 can be positioned remote from the system 100. Additionally, the operative note processing device 109 can receive the surgical procedure data (e.g., from the system 100) in real time or near real time and immediately process the surgical procedure data to generate the operative note. That is, the operative note processing device 109 can continuously populate the operative note with generated text that describes the surgical procedure as the surgical procedure proceeds. In other embodiments, the operative note processing device 109 can store the surgical procedure data as it is collected during the surgical procedure and/or receive the surgical procedure data after it has been collected during the surgical procedure. Then, after receipt of a user input or instruction after the surgical procedure is complete, the operative note processing device 109 can process the surgical procedure data to generate the operative note.

The various modules 440-448 of the operative note processing device operate together to carry out a method for automatically generating an operative note. The various modules 440-448 can be combined, implemented in the same or separate computing environments and/or in the same or different computing device, ordered differently, and/or selectively omitted. For example, in some embodiments the operative note processing device 441 optionally omits the data preprocessing module 441 and/or the interface module 448. Likewise, feedback and updates to the operative note can be implemented solely automatically (e.g., omitting the feedback module 447) solely manually (e.g., omitting the quality assurance and review module 446).

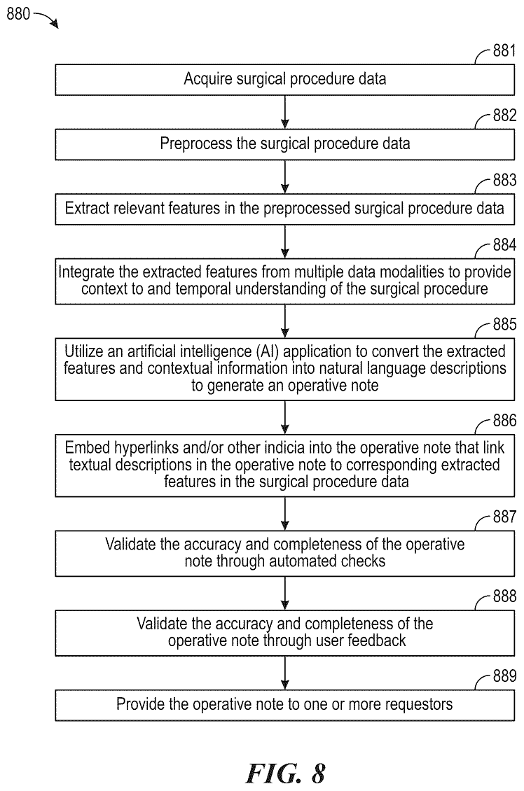

FIG. 8 is a flow diagram of a process or method 880 carried out by the operative note processing device 109 for automatically generating a surgical operative note in accordance with embodiments of the present technology. Although some features of the method 880 are described in the context of the embodiments shown in FIGS. 1-7D for the sake of illustration, one skilled in the art will readily understand that the method 880 can be carried out using other suitable systems and/or devices described herein.

At block 881, the method 880 can include acquiring surgical procedure data of surgical procedure. For example, as described in detail above with reference to the data acquisition module 440 of FIG. 4, the surgical procedure data can include multi-modal data including image, video, text, and/or other data captured intraoperatively (e.g., intraoperative video data captured by the cameras 112, the trackers 113, and/or the depth sensor 114) and/or preoperatively (e.g., preoperative CT and/or MRI images). The surgical procedure data can be acquired in real time or near real time during the surgical procedure, or can be received in full after completion of the surgical procedure.

At block 882, the method 880 can include preprocessing the surgical procedure data to enhance its quality, remove noise, and/or integrate different data modalities as, for example, described in detail above with reference to the data preprocessing module 441 of FIG. 4. At block 883, the method 880 can include extracting relevant features in the preprocessed surgical procedure data. As described in detail above with reference to the feature extraction module 442 of FIG. 4, computer vision techniques such as object detection, motion tracking, and/or image segmentation can be used to identify and extract surgical actions (e.g., blunt dissection, deep dissection, incision, closure, laminotomy), anatomical landmarks (e.g., spinous processes, inter-spinous ligaments, lamina, pars and facets), instrument movements (e.g., pedicle screw entry, cutting instrument usage, retractor usage), and intraoperative events (e.g., incision, dissection, closure).

At block 884, the method 880 can include integrating the extracted features from multiple data modalities to provide context to and temporal understanding of the surgical procedure. For example, as described in detail above with reference to the data fusion and contextual understanding module 443 of FIG. 4, the same features of the surgical procedure identified in different data modalities can be grouped together to provide a temporal understanding of the surgical procedure. Additionally, one or more AI applications can be used to provide contextual information about the features relevant to the surgical procedure.

At block 885, the method 880 can include utilizing an AI application to convert the extracted features and the related contextual information into natural language descriptions to generate an operative note. For example, as described in detail above with reference to the natural language generation module 444 of FIG. 4 and the operative note 550 of FIG. 5, the operative note is a structured and coherent description of the surgical procedure that summarizes the surgical procedure, including the type of surgery performed, specific surgical techniques used, intraoperative findings, postoperative care instructions, and/or the like.

At block 886, the method 880 can include embedding hyperlinks and/or other indicia into the operative note that link textual descriptions in the operative note to corresponding extracted features in the surgical procedure data. For example, as described in detail above with reference to the hyperlinking module 444 of FIG. 4 and the operative note 550 of FIGS. 6A-6E, the hyperlinks can allow a user viewing the operative note to quickly retrieve surgical procedure data (e.g., a video segment or image) corresponding to certain textual descriptions in the operative note.

At block 887, the method 880 can include validating the accuracy and completeness of the operative note through automated checks as, for example, described in detail above with reference to the quality assurance and review module 446 of FIG. 4. The operative note can be updated automatically to correct for any inaccuracies and/or to fill in omitted information. At block 888, the method 880 can include validating the accuracy and completeness of the operative note by soliciting user feedback. For example, as described in detail above with reference to the feedback module 447 of FIG. 4 and the operative note 550 of FIGS. 7A-7D, feedback indicators can be inserted into the operative note and selected by a user (e.g., a surgeon and/or surgical team member) to confirm or deny the accuracy of textual descriptions in the operative note. Any inaccuracies and/or omissions in the operative note can be corrected by the user. The updates to the operative note made at blocks 887 and 888 can be used as part of a reinforcement learning algorithm to update the models used by the AI applications and one or both of blocks 884 and 885.

Finally, at block 889, the method 880 can include providing the operative note can to one or more requestors. For example, as described in detail above with reference to the interface module 448 of FIG. 4, the method 880 can include providing the operative note to one or more (i) clinical health care systems for continued patient care, learning, training, etc., (ii) financial systems for verifying the medical necessity of the surgical procedure, ensuring appropriate coding, calculating the reimbursement amount based on established fee schedules or reimbursement rates, etc., and/or (iii) other interested parties (e.g., third party systems and/or applications).

Referring to FIGS. 1-8, in some aspects of the present technology the operative note processing device 109 can automatically generate an accurate surgical operative note describing a surgical procedure in a manner that provides improved efficiency, accuracy, standardization, and documentation compared to conventional manual methods for preparing operative notes. Regarding efficiency, the present technology can improve efficiency by automatically generating operative notes with no, reduced, and/or minimal effort on the part of a user (e.g., a surgeon or surgical team member). That is, the user need not manually prepare an operative note postoperatively and, at most, can simply provide select feedback to verify the accuracy of an automatically-generated operative note and/or to fill in any omissions therein. Regarding accuracy, the present technology can leverage AI algorithms and surgical data (e.g., video data) to produce accurate and detailed operative notes with minimal human intervention. Regarding standardization, the present technology can promote consistency and standardization in operative note documentation across surgical procedures and healthcare providers. Finally, regarding documentation, the present technology can capture rich, hyperlinked, and comprehensive information from surgical videos and other surgical procedure data, enhancing the quality and completeness of operative notes for clinical and medico-legal purposes. Accordingly, by addressing the limitations of traditional manual documentation methods, the present technology offers significant benefits in terms of efficiency, accuracy, standardization, and documentation, ultimately improving patient care and clinical workflow in surgical settings.

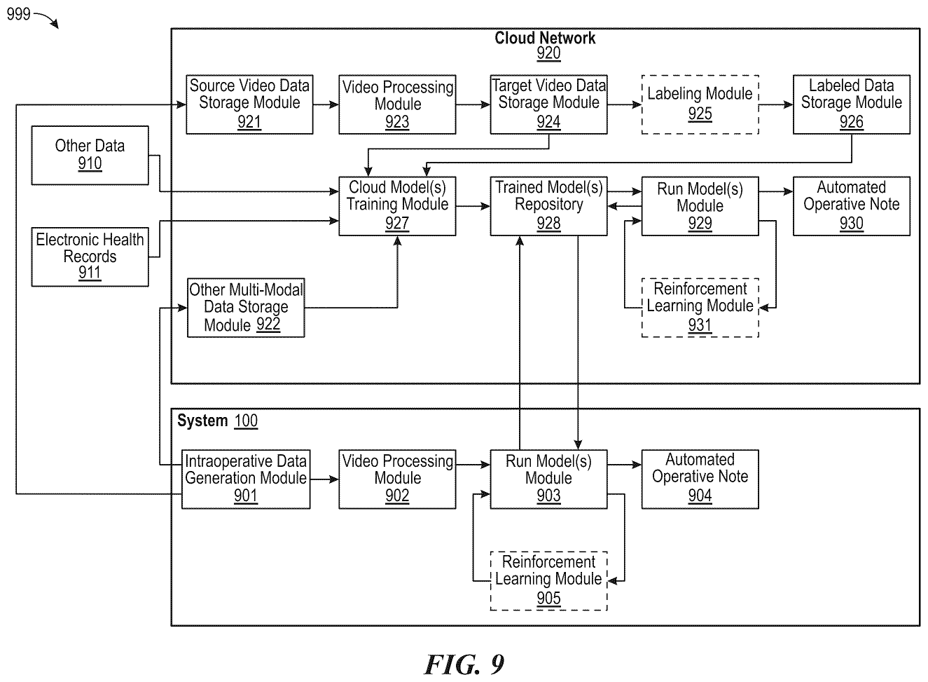

FIG. 9 is a block diagram of a functional computing environment 999 in which at least some operations described herein can be implemented in accordance with embodiments of the present technology. In the illustrated embodiment, the computing environment 999 includes the system 100 described in detail above with reference to FIGS. 1-8 communicatively coupled to a cloud network 920. In general, the cloud network 920 can be used to train and store one or more models (e.g., AI applications, machine learning models) configured to automatically generate an operative note as described in detail herein. The system 100 provides intraoperative data to the cloud network 920 that can be used to, for example, train the one or more models. The system 100 can also receive/access the one or more models from the cloud network 920 for automatically generating an operative note.

More specifically, in the illustrated embodiment the system 100 includes an intraoperative data generation module 901. As described in detail above, for example, with reference to the data acquisition module 440 of FIG. 4, the intraoperative data generation module 901 can generate, record, etc., intraoperative video data, tracking data, depth data, and/or the like from one or more video recording devices, depth cameras, endoscopes, and/or the like. For example, referring also to FIG. 1, the intraoperative data generation module 901 can record video data from the cameras 112 (e.g., RGB video data), video data from the trackers 113 (e.g., infrared video data), depth data from the depth sensor 114, etc., during a surgical procedure. The system 100 can further include a video processing module 902 configured to receive video data and/or other data from the intraoperative data generation module 901 and to process the video data and/or other data. In some embodiments, the video processing module 902 can function similarly or identically to the data preprocessing module 441 described in detail above with reference to FIG. 4 to, for example, process the data to enhance its quality, remove noise, and/or integrate different data modalities.

In the illustrated embodiment, the cloud network 920 includes a source video data storage module 921 configured to receive and store intraoperative video data generated by the intraoperative data generation module 901, and an other multi-modal data storage module 922 configured to receive non-video modalities of data (e.g., tracking data, depth data, registration data, navigation data, audio data) generated by the intraoperative data generation module 901. The cloud network 920 can further include a video processing module 923 that can operate similarly or identically to the video processing module 902 of the system 100, and a target video data storage module 924 configured to receive and store target video data processed by the video processing module 923. The cloud network 920 can further include a labeling module 925 configured to receive input from a clinician, surgeon, and/or the like labeling some or all of the target video data from the target video data storage module 924. The labeling can include assigning labels, tags, annotations, and/or the like to the target video data that, for example, identify different features in the target video data such as surgical events, surgical actions, and/or the like. Accordingly, in some embodiments the labels correspond to the features that are aimed to be identified by the feature extraction module 442 of FIG. 4, and provide a benchmark (e.g., ground truth, correct answer) for the subsequent model training. A labeled data storage module 926 can received the labeled data from the labeling module 925 and store the labeled data for subsequent use in model training, testing, and validation.

In the illustrated embodiment, the cloud network 920 further includes a cloud model(s) training module 927 configured to receive (i) the target video data from the target video data storage module 924, (ii) the labeled data from the labeled data storage module 926, (iii) other data 910, (iv) electronic health records 911 (e.g., preoperative CT data, preoperative MRI data), and (v) other multi-modal data from the other multi-modal data storage module 922. The other data 910 and the electronic health records 911 can be generated and stored outside the system 100. The cloud model(s) training module 927 is configured to train one or more AI applications or models (e.g., a machine learning model) based on the different data sets to, for example, carry out the functions of automatically generating an operative note as described in detail above with reference to FIGS. 4-8. Specifically, the cloud model(s) training module 927 can carry out a supervised learning process in which the model is trained on the labeled data from the labeled data storage module 926, the model's performance is evaluated, the model is validated and tested based on one or more unlabeled data sets (e.g., the target video data from the target video data storage module 924, the other data 910, the electronic health records 911, and/or the other multi-modal data from the other multi-modal data storage module 922), errors are corrected, and the model is finalized. The cloud model(s) training module 927 can output the one or more trained AI applications or models for storage in a trained model(s) repository 928.

In some embodiments, the system 100 includes a run model(s) module 903 configured to access the trained model(s) repository 928 to access one or more of the trained models to run the trained model(s) based on processed video data input from the video processing module 902 to automatically generate an operative note 904 as described in detail above with reference to FIGS. 4-8. In some embodiments, the system 100 further includes a reinforcement learning module 905 configured to receive input from a clinician, surgeon, and/or the like regarding a quality of the automatically generated operative note 904 and to update and/or fine-tune the model parameters as, for example, described in detail above with reference to FIG. 4. The updated model after reinforcement learning can be sent to the trained model(s) repository 928 for storage. Alternatively or additionally, the trained model(s) can be run on the cloud network 920 rather than the system 100 via a run model(s) module 929 to generate an automated operative note 930. The cloud network 920 can likewise include a reinforcement learning module 931 configured to receive input for reinforcement learning and updating of the model parameters.

### III. Selected Embodiments of Systems and Methods for Generating a Surgical Description Other than an Operative Note

In some embodiments, some aspects of the present technology can be utilized to automatically generate a surgical description other than an operative note. For example, aspects of the operative note processing device 109 (FIG. 4) can be used to automatically extract features and generate natural language descriptions of a surgical procedure for clinical educational use, such as for generating a PowerPoint or other summary of a surgical procedure summarizing key aspects of the surgical procedure and displaying accompanying images and/or videos. For example, the present technology can utilize one or more artificial intelligence (AI) applications to extract features and context from various preoperative, intraoperative, and/or postoperative data streams to generate a summary of the surgical procedure including aspects like preoperative and postoperative imaging, descriptions of the preoperative pathology, intraoperative and postoperative alignment and resultant parameters, clips of key surgical actions, instrument and hardware usage, surgical techniques used, and/or the like. Such presentations, commonly generated manually in PowerPoint by surgeons or other healthcare team members, are a critical aspect of clinical education, training, and improvement. By automating the existing manual process, the present technology can reduce healthcare time constraints and improve clinical education and outcomes.

### IV. Selected Embodiments of Computing Environments

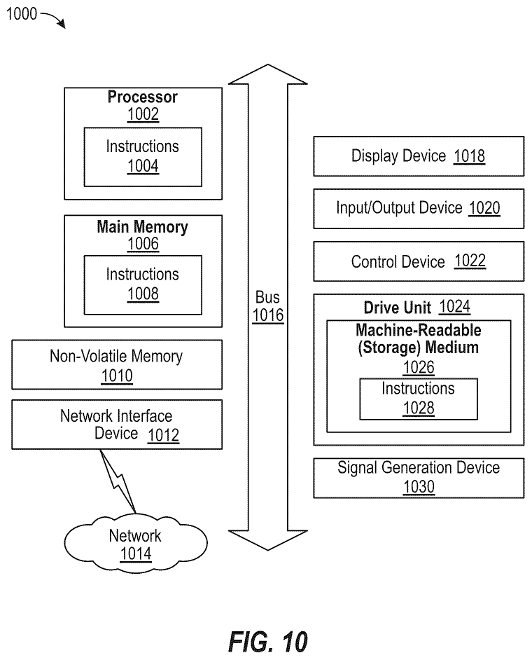

FIG. 10 is a block diagram that illustrates an example of a computer system 1000 in which at least some operations described herein can be implemented. The computer system 1000 can include: one or more processors 1002, a main memory 1006, a non-volatile memory 1010, a network interface device 1012, a display device 1018, an input/output device 1020, a control device 1022 (e.g., keyboard and pointing device), a drive unit 1024 that includes a machine readable (storage) medium 1026, and a signal generation device 1030 that are communicatively connected to a bus 1016. The bus 1016 represents one or more physical buses and/or point-to-point connections that are connected by appropriate bridges, adapters, and/or controllers. Various common components (e.g., cache memory) are omitted from FIG. 10 for brevity. Instead, the computer system 1000 is intended to illustrate a hardware device on which components illustrated or described relative to the examples of the figures and any other components described in this specification can be implemented.

The computer system 1000 can take any suitable physical form. For example, the computer system 1000 can share a similar architecture as that of a server computer, personal computer (PC), tablet computer, mobile telephone, wearable electronic device, network-connected (“smart”) device (e.g., a television or home assistant device), AR/VR system (e.g., head-mounted display), or any electronic device capable of executing a set of instructions that specify action(s) to be taken by the computer system 1000. In some implementations, the computer system 1000 can be an embedded computer system, a system-on-chip (SOC), a single-board computer (SBC) system, or a distributed system such as a mesh of computer systems or include one or more cloud components in one or more networks. Where appropriate, one or more computer systems 1000 can perform operations in real time, near real time, or in batch mode.

The network interface device 1012 enables the computer system 1000 to mediate data in a network 1014 with an entity that is external to the computer system 1000 through any communication protocol supported by the computer system 1000 and the external entity. Examples of the network interface device 1012 include a network adapter card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., the main memory 1006, the non-volatile memory 1010, the machine-readable medium 1026) can be local, remote, or distributed. Although shown as a single medium, the machine-readable medium 1026 can include multiple media (e.g., a centralized/distributed database and/or associated caches and servers) that store one or more sets of instructions 1028. The machine-readable medium 1026 can include any medium that is capable of storing, encoding, or carrying a set of instructions for execution by the computer system 1000. The machine-readable medium 1026 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 1010, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 1004, 1008, 1028) set at various times in various memory and storage devices in computing device(s). When read and executed by the processor 1002, the instruction(s) cause the computer system 1000 to perform operations to execute elements involving the various aspects of the disclosure.

### V. Selected Embodiments of Artificial Intelligence and Machine Learning Implementations

To assist in understanding the present disclosure, some concepts relevant to neural networks and machine learning (ML) are discussed herein. Generally, a neural network comprises a number of computation units (sometimes referred to as “neurons”). Each neuron receives an input value and applies a function to the input to generate an output value. The function typically includes a parameter (also referred to as a “weight”) whose value is learned through the process of training. A plurality of neurons may be organized into a neural network layer (or simply “layer”) and there may be multiple such layers in a neural network. The output of one layer may be provided as input to a subsequent layer. Thus, input to a neural network may be processed through a succession of layers until an output of the neural network is generated by a final layer. This is a simplistic discussion of neural networks and there may be more complex neural network designs that include feedback connections, skip connections, and/or other such possible connections between neurons and/or layers, which are not discussed in detail here.

A deep neural network (DNN) is a type of neural network having multiple layers and/or a large number of neurons. The term DNN can encompass any neural network having multiple layers, including convolutional neural networks (CNNs), recurrent neural networks (RNNs), multilayer perceptrons (MLPs), Generative Adversarial Networks (GANs), Variational Autoencoders (VAEs), and Auto-regressive Models, among others.

DNNs are often used as ML-based models for modeling complex behaviors (e.g., human language, image recognition, object classification, etc.) in order to improve the accuracy of outputs (e.g., more accurate predictions) such as, for example, compared with models with fewer layers. In the present disclosure, the term “ML-based model” or more simply “ML model” may be understood to refer to a DNN. Training an ML model refers to a process of learning the values of the parameters (or weights) of the neurons in the layers such that the ML model is able to model the target behavior to a desired degree of accuracy. Training typically requires the use of a training dataset, which is a set of data that is relevant to the target behavior of the ML model.

As an example, to train an ML model that is intended to model human language (also referred to as a “language model”), the training dataset may be a collection of text documents, referred to as a “text corpus” (or simply referred to as a “corpus”). The corpus may represent a language domain (e.g., a single language), a subject domain (e.g., scientific papers), and/or may encompass another domain or domains, be they larger or smaller than a single language or subject domain. For example, a relatively large, multilingual, and non-subject-specific corpus can be created by extracting text from online webpages and/or publicly available social media posts. Training data can be annotated with ground truth labels (e.g., each data entry in the training dataset can be paired with a label) or may be unlabeled.

Training an ML model generally involves inputting into an ML model (e.g., an untrained ML model) training data to be processed by the ML model, processing the training data using the ML model, collecting the output generated by the ML model (e.g., based on the inputted training data), and comparing the output to a desired set of target values. If the training data is labeled, the desired target values may be, for example, the ground truth labels of the training data. If the training data is unlabeled, the desired target value may be a reconstructed (or otherwise processed) version of the corresponding ML model input (e.g., in the case of an autoencoder), or can be a measure of some target observable effect on the environment (e.g., in the case of a reinforcement learning agent). The parameters of the ML model are updated based on a difference between the generated output value and the desired target value. For example, if the value outputted by the ML model is excessively high, the parameters may be adjusted so as to lower the output value in future training iterations. An objective function is a way to quantitatively represent how close the output value is to the target value. An objective function represents a quantity (or one or more quantities) to be optimized (e.g., minimize a loss or maximize a reward) in order to bring the output value as close to the target value as possible. The goal of training the ML model typically is to minimize a loss function or maximize a reward function.

The training data can be a subset of a larger data set. For example, a data set may be split into three mutually exclusive subsets: a training set, a validation (or cross-validation) set, and a testing set. The three subsets of data may be used sequentially during ML model training. For example, the training set may be first used to train one or more ML models, each ML model, for example, having a particular architecture, having a particular training procedure, being describable by a set of model hyperparameters, and/or otherwise being varied from the other of the one or more ML models. The validation (or cross-validation) set may then be used as input data into the trained ML models to, for example, measure the performance of the trained ML models and/or compare performance between them. Where hyperparameters are used, a new set of hyperparameters can be determined based on the measured performance of one or more of the trained ML models, and the first step of training (e.g., with the training set) may begin again on a different ML model described by the new set of determined hyperparameters. In this way, these steps can be repeated to produce a more performant trained ML model. Once such a trained ML model is obtained (e.g., after the hyperparameters have been adjusted to achieve a desired level of performance), a third step of collecting the output generated by the trained ML model applied to the third subset (the testing set) may begin. The output generated from the testing set may be compared with the corresponding desired target values to give a final assessment of the trained ML model's accuracy. Other segmentations of the larger data set and/or schemes for using the segments for training one or more ML models are possible.

Backpropagation is an algorithm for training an ML model. Backpropagation is used to adjust (e.g., update) the value of the parameters in the ML model, with the goal of optimizing the objective function. For example, a defined loss function is calculated by forward propagation of an input to obtain an output of the ML model and a comparison of the output value with the target value. Backpropagation calculates a gradient of the loss function with respect to the parameters of the ML model, and a gradient algorithm (e.g., gradient descent) is used to update (e.g., “learn”) the parameters to reduce the loss function. Backpropagation is performed iteratively so that the loss function is converged or minimized. Other techniques for learning the parameters of the ML model can be used. The process of updating (or learning) the parameters over many iterations is referred to as training. Training may be carried out iteratively until a convergence condition is met (e.g., a predefined maximum number of iterations has been performed, or the value outputted by the ML model is sufficiently converged with the desired target value), after which the ML model is considered to be sufficiently trained. The values of the learned parameters can then be fixed and the ML model may be deployed to generate output in real-world applications (also referred to as “inference”).

In some examples, a trained ML model may be fine-tuned, meaning that the values of the learned parameters may be adjusted slightly in order for the ML model to better model a specific task. Fine-tuning of an ML model typically involves further training the ML model on a number of data samples (which may be smaller in number/cardinality than those used to train the model initially) that closely target the specific task. For example, an ML model for generating natural language that has been trained generically on publicly available text corpora may be, for example, fine-tuned by further training using specific training samples. The specific training samples can be used to generate language in a certain style or in a certain format. For example, the ML model can be trained to generate a blog post having a particular style and structure with a given topic.

Some concepts in ML-based language models are now discussed. It may be noted that, while the term “language model” has been commonly used to refer to an ML-based language model, there could exist non-ML language models. In the present disclosure, the term “language model” can refer to an ML-based language model (e.g., a language model that is implemented using a neural network or other ML architecture), unless stated otherwise. For example, unless stated otherwise, the “language model” encompasses LLMs.

A language model can use a neural network (typically a DNN) to perform natural language processing (NLP) tasks. A language model can be trained to model how words relate to each other in a textual sequence, based on probabilities. A language model may contain hundreds of thousands of learned parameters or, in the case of an LLM, can contain millions or billions of learned parameters or more. As non-limiting examples, a language model can generate text, translate text, summarize text, answer questions, write code (e.g., Python, JavaScript, or other programming languages), classify text (e.g., to identify spam emails, to identify unintelligible inputs), create content for various purposes (e.g., social media content, factual content, or marketing content), and/or create personalized content for a particular individual or group of individuals. Language models can also be used for chatbots (e.g., virtual assistance).

A type of neural network architecture, referred to as a “transformer,” can be used for language models. For example, the Bidirectional Encoder Representations from Transformers (BERT) model, the Transformer-XL model, and the Generative Pre-trained Transformer (GPT) models are types of transformers. A transformer is a type of neural network architecture that uses self-attention mechanisms in order to generate predicted output based on input data that has some sequential meaning (i.e., the order of the input data is meaningful, which is the case for most text input). Although transformer-based language models are described herein, it should be understood that the present disclosure may be applicable to any ML-based language model, including language models based on other neural network architectures such as recurrent neural network (RNN)-based language models.

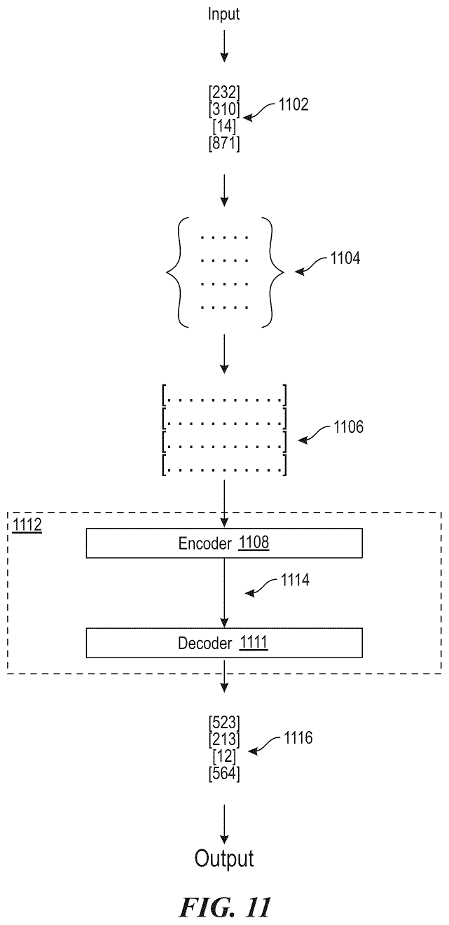

FIG. 11 is a block diagram of an example transformer 1112. A transformer is a type of neural network architecture that uses self-attention mechanisms to generate predicted output based on input data that has some sequential meaning (e.g., the order of the input data is meaningful, which is the case for most text input). Self-attention is a mechanism that relates different positions of a single sequence to compute a representation of the same sequence. Although transformer-based language models are described herein, the present disclosure may be applicable to any ML-based language model, including language models based on other neural network architectures such as recurrent neural network (RNN)-based language models.

The transformer 1112 includes an encoder 1108 (which can include one or more encoder layers/blocks connected in series) and a decoder 1110 (which can include one or more decoder layers/blocks connected in series). Generally, the encoder 1108 and the decoder 1110 each include multiple neural network layers, at least one of which can be a self-attention layer. The parameters of the neural network layers can be referred to as the parameters of the language model.

The transformer 1112 can be trained to perform certain functions on a natural language input. Examples of the functions include summarizing existing content, brainstorming ideas, writing a rough draft, fixing spelling and grammar, translating content, and/or the functions attributed to various artificial intelligence (AI) applications described in detail above with reference to FIGS. 1-9. Summarizing can include extracting key points or themes from an existing content in a high-level summary. Brainstorming ideas can include generating a list of ideas based on provided input. For example, the ML model can generate a list of names for a startup or costumes for an upcoming party. Writing a rough draft can include generating writing in a particular style that could be useful as a starting point for the user's writing. The style can be identified as, e.g., an email, a blog post, a social media post, or a poem. Fixing spelling and grammar can include correcting errors in an existing input text. Translating can include converting an existing input text into a variety of different languages. In some implementations, the transformer 1112 is trained to perform certain functions on other input formats than natural language input. For example, the input can include objects, images, audio content, or video content, or a combination thereof.

The transformer 1112 can be trained on a text corpus that is labeled (e.g., annotated to indicate verbs, nouns) or unlabeled. LLMs can be trained on a large unlabeled corpus. The term “language model,” as used herein, can include an ML-based language model (e.g., a language model that is implemented using a neural network or other ML architecture), unless stated otherwise. Some LLMs can be trained on a large multi-language, multi-domain corpus to enable the model to be versatile at a variety of language-based tasks such as generative tasks (e.g., generating human-like natural language responses to natural language input).

FIG. 11 illustrates an example of how the transformer 1112 can process textual input data. Input to a language model (whether transformer-based or otherwise) typically is in the form of natural language that can be parsed into tokens. The term “token” in the context of language models and NLP has a different meaning from the use of the same term in other contexts such as data security. Tokenization, in the context of language models and NLP, refers to the process of parsing textual input (e.g., a character, a word, a phrase, a sentence, a paragraph) into a sequence of shorter segments that are converted to numerical representations referred to as tokens (or “compute tokens”). Typically, a token can be an integer that corresponds to the index of a text segment (e.g., a word) in a vocabulary dataset. Often, the vocabulary dataset is arranged by frequency of use. Commonly occurring text, such as punctuation, can have a lower vocabulary index in the dataset and thus be represented by a token having a smaller integer value than less commonly occurring text. Tokens frequently correspond to words, with or without white space appended. In some implementations, a token can correspond to a portion of a word.

For example, the word “greater” can be represented by a token for [great] and a second token for [er]. In another example, the text sequence “write a summary” can be parsed into the segments [write], [a], and [summary], each of which can be represented by a respective numerical token. In addition to tokens that are parsed from the textual sequence (e.g., tokens that correspond to words and punctuation), there can also be special tokens to encode non-textual information. For example, a [CLASS] token can be a special token that corresponds to a classification of the textual sequence (e.g., can classify the textual sequence as a list, a paragraph), an [EOT] token can be another special token that indicates the end of the textual sequence, other tokens can provide formatting information, etc.

In FIG. 11, a short sequence of tokens 1102 corresponding to the input text is illustrated as input to the transformer 1112. Tokenization of the text sequence into the tokens 1102 can be performed by some pre-processing tokenization module such as, for example, a byte-pair encoding tokenizer (the “pre” referring to the tokenization occurring prior to the processing of the tokenized input by the LLM), which is not shown in FIG. 11 for brevity. In general, the token sequence that is inputted to the transformer 1112 can be of any length up to a maximum length defined based on the dimensions of the transformer 1112. Each token 1102 in the token sequence is converted into an embedding vector 1106 (also referred to as “embedding 1106”).

An embedding 1106 is a learned numerical representation (such as, for example, a vector) of a token that captures some semantic meaning of the text segment represented by the token 1102. The embedding 1106 represents the text segment corresponding to the token 1102 in a way such that embeddings corresponding to semantically related text are closer to each other in a vector space than embeddings corresponding to semantically unrelated text. For example, assuming that the words “write,” “a,” and “summary” each correspond to, respectively, a “write” token, an “a” token, and a “summary” token when tokenized, the embedding 1106 corresponding to the “write” token will be closer to another embedding corresponding to the “jot down” token in the vector space as compared to the distance between the embedding 1106 corresponding to the “write” token and another embedding corresponding to the “summary” token.

The vector space can be defined by the dimensions and values of the embedding vectors. Various techniques can be used to convert a token 1102 to an embedding 1106. For example, another trained ML model can be used to convert the token 1102 into an embedding 1106. In particular, another trained ML model can be used to convert the token 1102 into an embedding 1106 in a way that encodes additional information into the embedding 1106 (e.g., a trained ML model can encode positional information about the position of the token 1102 in the text sequence into the embedding 206). In some implementations, the numerical value of the token 1102 can be used to look up the corresponding embedding in an embedding matrix 1104, which can be learned during training of the transformer 1112.

The generated embeddings 1106 are input into the encoder 1108. The encoder 1108 serves to encode the embeddings 1106 into feature vectors 1114 that represent the latent features of the embeddings 1106. The encoder 1108 can encode positional information (i.e., information about the sequence of the input) in the feature vectors 1114. The feature vectors 1114 can have very high dimensionality (e.g., on the order of thousands or tens of thousands), with each element in a feature vector 1114 corresponding to a respective feature. The numerical weight of each element in a feature vector 1114 represents the importance of the corresponding feature. The space of all possible feature vectors 1114 that can be generated by the encoder 1108 can be referred to as a latent space or feature space.

Conceptually, the decoder 1110 is designed to map the features represented by the feature vectors 1114 into meaningful output, which can depend on the task that was assigned to the transformer 1112. For example, if the transformer 1112 is used for a translation task, the decoder 1110 can map the feature vectors 1114 into text output in a target language different from the language of the original tokens 1102. Generally, in a generative language model, the decoder 1110 serves to decode the feature vectors 1114 into a sequence of tokens. The decoder 1110 can generate output tokens 1116 one by one. Each output token 1116 can be fed back as input to the decoder 1110 in order to generate the next output token 1116. By feeding back the generated output and applying self-attention, the decoder 1110 can generate a sequence of output tokens 1116 that has sequential meaning (e.g., the resulting output text sequence is understandable as a sentence and obeys grammatical rules). The decoder 1110 can generate output tokens 1116 until a special [EOT] token (indicating the end of the text) is generated. The resulting sequence of output tokens 1116 can then be converted to a text sequence in post-processing. For example, each output token 1116 can be an integer number that corresponds to a vocabulary index. By looking up the text segment using the vocabulary index, the text segment corresponding to each output token 1116 can be retrieved, the text segments can be concatenated together, and the final output text sequence can be obtained.

In some implementations, the input provided to the transformer 1112 includes instructions to perform a function on an existing text. The output can include, for example, a modified version of the input text and instructions to modify the text. The modification can include summarizing, translating, correcting grammar or spelling, changing the style of the input text, lengthening or shortening the text, or changing the format of the text (e.g., adding bullet points or checkboxes). As an example, the input text can include meeting notes prepared by a user and the output can include a high-level summary of the meeting notes. In other examples, the input provided to the transformer includes a question or a request to generate text. The output can include a response to the question, text associated with the request, or a list of ideas associated with the request. For example, the input can include the question “What is the weather like in San Francisco?” and the output can include a description of the weather in San Francisco. As another example, the input can include a request to brainstorm names for a flower shop and the output can include a list of relevant names.

Although a general transformer architecture for a language model and its theory of operation have been described above, this is not intended to be limiting. Existing language models include language models that are based only on the encoder of the transformer or only on the decoder of the transformer. An encoder-only language model encodes the input text sequence into feature vectors that can then be further processed by a task-specific layer (e.g., a classification layer). BERT is an example of a language model that can be considered to be an encoder-only language model. A decoder-only language model accepts embeddings as input and can use auto-regression to generate an output text sequence. Transformer-XL and GPT-type models can be language models that are considered to be decoder-only language models.

Because GPT-type language models tend to have a large number of parameters, these language models can be considered LLMs. An example of a GPT-type LLM is GPT-3. GPT-3 is a type of GPT language model that has been trained (in an unsupervised manner) on a large corpus derived from documents available online to the public. GPT-3 has a very large number of learned parameters (on the order of hundreds of billions), can accept a large number of tokens as input (e.g., up to 2,048 input tokens), and is able to generate a large number of tokens as output (e.g., up to 2,048 tokens). GPT-3 has been trained as a generative model, meaning that it can process input text sequences to predictively generate a meaningful output text sequence. ChatGPT is built on top of a GPT-type LLM and has been fine-tuned with training datasets based on text-based chats (e.g., chatbot conversations). ChatGPT is designed for processing natural language, receiving chat-like inputs, and generating chat-like outputs.

A computer system can access a remote language model (e.g., a cloud-based language model), such as ChatGPT or GPT-3, via a software interface (e.g., an API). Additionally or alternatively, such a remote language model can be accessed via a network such as the Internet. In some implementations, such as, for example, potentially in the case of a cloud-based language model, a remote language model can be hosted by a computer system that can include a plurality of cooperating (e.g., cooperating via a network) computer systems that can be in, for example, a distributed arrangement. Notably, a remote language model can employ multiple processors (e.g., hardware processors such as, for example, processors of cooperating computer systems). Indeed, processing of inputs by an LLM can be computationally expensive/can involve a large number of operations (e.g., many instructions can be executed/large data structures can be accessed from memory), and providing output in a required timeframe (e.g., real time or near real time) can require the use of a plurality of processors/cooperating computing devices as discussed above.

Inputs to an LLM can be referred to as a prompt, which is a natural language input that includes instructions to the LLM to generate a desired output. A computer system can generate a prompt that is provided as input to the LLM via an API. As described above, the prompt can optionally be processed or pre-processed into a token sequence prior to being provided as input to the LLM via its API. A prompt can include one or more examples of the desired output, which provides the LLM with additional information to enable the LLM to generate output according to the desired output. Additionally or alternatively, the examples included in a prompt can provide inputs (e.g., example inputs) corresponding to/as can be expected to result in the desired outputs provided. A one-shot prompt refers to a prompt that includes one example, and a few-shot prompt refers to a prompt that includes multiple examples. A prompt that includes no examples can be referred to as a zero-shot prompt.

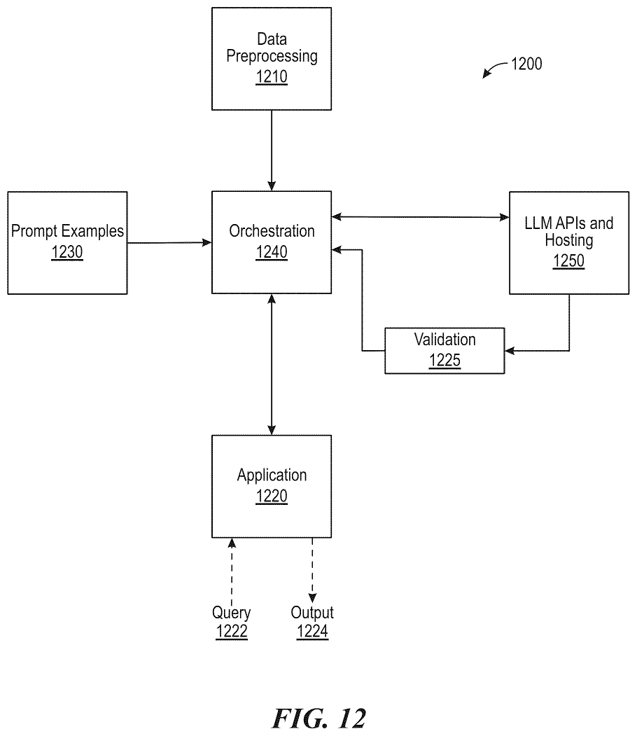

FIG. 12 is a block diagram illustrating an architecture 1200 for LLM applications, according to some implementations. As shown in FIG. 12, the architecture 1200 can include a data preprocessing block 1210, an application 1220, a prompt examples block 1230, an orchestration block 1240, an LLM APIs and Hosting block 1250, and a validation block 1255. Other implementations of the architecture 1200 can include additional, fewer, or different components, or can distribute functionality differently among the components.

The data preprocessing block 1210 manages contextual data and embeddings that can be used to train LLMs or to serve as a data source for an LLM to generate an output. Contextual data can include documents in any of a variety of formats, including text, PDFs, SQL tables, CSV files, images, or code repositories. The data preprocessing block 1210 can retrieve the contextual data from publicly available sources, private sources associated with the application 1220, or a combination of public and private sources.

The data preprocessing block 1210 can generate embeddings of the contextual data or invoke a service to generate the embeddings. The models used to generate embeddings can be trained for the specific model or application in which the embeddings are to be used. Embeddings can be stored in a vector database.

An application 1220 interfaces between a user or external system and the architecture of the LLM. A query 1222 can be input at the application 1220. Based on the query, the application 1220 generates a prompt or series of prompts to cause the LLM to produce a specified output. The application 1220 returns outputs 1224 from the LLM to the requesting user or system.

A prompt is an input to an LLM that instructs the LLM to generate a desired output. Prompts can be structured as a natural language input that includes elements of a user query, hardcoded or dynamically generated prompts templates, data retrieved from external sources at the time the prompt is generated, or other elements that provide contextual data, specific instructions, or validation requirements for the LLM. A computing system, such as the application 1220, generates a prompt that is provided as input to the LLM via the LLM's API. As described above, the prompt may optionally be processed or pre-processed into a token sequence prior to being provided as input to the LLM.

Some prompts can include one or more examples of the desired output, which provides the LLM with additional information to enable the LLM to better generate output according to the desired output. Additionally or alternatively, the examples included in a prompt may provide inputs (e.g., example inputs) corresponding to/as may be expected to result in the desired outputs provided. A one-shot prompt refers to a prompt that includes one example, and a few-shot prompt refers to a prompt that includes multiple examples. A prompt that includes no examples can be referred to as a zero-shot prompt. The prompt examples block 1230 provides these example outputs to the LLM for one-shot or few-shot prompts. Example outputs can be provided to the prompt examples block 1230 by a user or developer of the application 1220, in some cases.

The orchestration block 1240 interfaces between LLM application programming interfaces (APIs), the data preprocessing block 1210, the application 1220, the prompt examples block 1230, and/or other data sources or systems. The orchestration block 1240 can submits prompts received from the application 1220 to the LLM. In some implementations, the orchestration block 1240 causes the prompt to be pre-processed into a token sequence prior to being provided as input to the LLM. The orchestration block 1240 can also process prompts to prioritize embeddings that are more relevant to produce a particular output from the LLM or to reorder prompts or embeddings to enable the LLM to produce a contextually relevant response.

The validation block 1255 validates outputs from the LLM before providing the outputs to the requesting application 1220.

In some embodiments, aspects of the present technology can utilize a two-stage temporal convolutional model. The two-stage temporal convolutional model can be of the type described in (i) “Surgical workflow recognition with temporal convolution and transformer for action segmentation,” published in the International of Computer Assisted Radiology and Surgery, by B. Zhang, B. Goel, M. H. Sarhan, V. K. Goel, R. Abukhalil, B. Kalesan, N. Stottler, and S. Petculescu, 2023; 18(4):785-794. doi:10.1007/s11548-022-02811-z, and available at https://pubmed.ncbi.nlm.nih.gov/36542253/ and/or (ii) “MS-TCN++: Multi-Stage Temporal Convolutional Network for Action Segmentation,” published in IEEE Transactions on Pattern Analysis and Machine Intelligence, by S. Li, Y. A. Farha, Y. Liu, M.-M. Cheng, and J. Gall, vol. 45, no. 6, pp. 6647-6658, 1 Jun. 2023, doi:10.1109/TPAMI.2020.3021756, and available at https://ieeexplore.ieee.org/document/9186840, each of which is hereby incorporated by reference in its entirety.

A two-stage temporal convolutional model is a type of deep learning architecture designed to process sequential data (such as time series, video, or audio) by capturing temporal dependencies using convolutional layers. The “two-stage” aspect refers to the model being divided into two distinct processing phases, each with a specific role. The model first extracts broad temporal features, then refines them for accurate, temporally consistent predictions, making it highly effective for sequential data analysis.

The first stage provides an initial temporal feature extraction. The purpose of the first stage is to extract coarse temporal features from the input sequence. During the first stage, the input sequence (e.g., a series of video frames or sensor readings) is passed through a stack of temporal convolutional layers. The convolutional layers use one-dimensional (1D) convolutions across the time dimension, allowing the model to learn patterns and dependencies over time. The output is a set of feature maps that summarize the temporal structure of the input. For example, for action segmentation in videos, the first stage can identify rough boundaries of different actions.

The second stage refines the features identified by the first stage, correcting errors and/or improving temporal consistency. During the second stage, the features output from the first stage are input into another set of temporal convolutional layers. The second stage focuses on fine-grained temporal relationships and can correct over-segmentation or smooth out predictions. The final output of the second stage is typically a sequence of class labels, probabilities, or other predictions aligned with the input sequence. For example, for action segmentation in videos, the second stage can refine the action boundaries and ensures that the predicted actions are temporally coherent.

### EXAMPLES

The following examples are illustrative of several embodiments of the present technology:

1. A method of generating an operative note for a surgical procedure, the method comprising:
receiving surgical procedure data of the surgical procedure;
identifying features in the surgical procedure data relevant to the surgical procedure;
processing the identified features to provide contextual information about the surgical procedure; and
utilizing an artificial intelligence (AI) application to generate the operative note based on the identified features and the contextual information, wherein the operative note includes a natural language description of the surgical procedure.

2. The method of example 1 wherein the method further comprises embedding a hyperlink in the operative note, wherein the hyperlink links a textual description in the operative note to a corresponding feature identified in the surgical procedure data.

3. The method of example 2 wherein the corresponding feature identified in the surgical procedure comprises a video segment.

4. The method of any one of examples 1-3 wherein the method further comprises automatically validating the accuracy and completeness of the operative note.

5. The method of example 4 wherein the method further comprises utilizing data related to the automatic validation to train the AI application via reinforcement learning.

6. The method of any one of examples 1-5 wherein the method further comprises:
soliciting user feedback to validate the accuracy and completeness of the operative note; and
receiving the user feedback.

7. The method of example 6 wherein the method further comprises utilizing the user feedback to train the AI application via reinforcement learning.

8. The method of any one of examples 1-7 wherein the surgical procedure data comprises multiple modalities of data.

9. The method of any one of examples 1-8 wherein the surgical procedure data comprises intraoperative video data of the surgical procedure.

10. The method of any one of examples 1-9 wherein the surgical procedure is a spinal surgical procedure.

11. A system for generating an operative note for a surgical procedure, the system comprising:
a sensor array including multiple sensors configured to capture surgical procedure data of the surgical procedure; and
an operative note generation device programmed with non-transitory computer readable instructions that, when executed by the operative note generation device, cause the operative note generation device to—
   acquire the surgical procedure data captured by the sensor array;
   identify features in the surgical procedure data relevant to the surgical procedure;
   process the identified features to provide contextual information about the surgical procedure; and
   utilize an artificial intelligence (AI) application to generate the operative note based on the identified features and the contextual information, wherein the operative note includes a natural language description of the surgical procedure.

12. The system of example 11 wherein the operative note generation device is positioned local to the sensor array.

13. The system of example 11 or example 12 wherein the operative note generation device is positioned remote from the sensor array.

14. The system of any one of examples 11-13 wherein the multiple sensors include RGB cameras, and wherein the surgical procedure data comprises RGB image data.

15. The system of any one of examples 11-14 wherein the computer readable instructions, when executed by the operative note generation device, cause the operative note generation device to acquire the surgical procedure data in real time or near real time from the sensor array.

16. The system of any one of examples 11-15 wherein the computer readable instructions, when executed by the operative note generation device, further cause the operative note generation device to:
acquire additional data related to the surgical procedure from a source other than the sensor array; and
process the identified features and the additional data to provide contextual information about the surgical procedure.

17. The system of example 16 wherein the additional data comprises preoperative image data of a patient undergoing the surgical procedure.

18. The system of any one of examples 11-17 wherein the computer readable instructions, when executed by the operative note generation device, further cause the operative note generation device to embed a hyperlink in the operative note, wherein the hyperlink links a textual description in the operative note to a corresponding feature identified in the surgical procedure data.

19. The system of example 18 wherein the corresponding feature identified in the surgical procedure comprises a video segment.

20. A method of generating an operative note for a surgical procedure, the method comprising:
capturing surgical procedure data of the surgical procedure with a sensor array positioned to view the surgical procedure;
identifying features in the surgical procedure data relevant to the surgical procedure;
processing the identified features to provide contextual information about the surgical procedure; and
utilizing an artificial intelligence (AI) application to generate the operative note based on the identified features and the contextual information, wherein the operative note includes a natural language description of the surgical procedure.

### VII. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method of generating an operative note for a surgical procedure, the method comprising:
receiving surgical procedure data of the surgical procedure;
identifying features in the surgical procedure data relevant to the surgical procedure;
processing the identified features to provide contextual information about the surgical procedure; and
utilizing an artificial intelligence (AI) application to generate the operative note based on the identified features and the contextual information, wherein the operative note includes a natural language description of the surgical procedure.

2. The method of claim 1 wherein the method further comprises embedding a hyperlink in the operative note, wherein the hyperlink links a textual description in the operative note to a corresponding feature identified in the surgical procedure data.

3. The method of claim 2 wherein the corresponding feature identified in the surgical procedure comprises a video segment.

4. The method of claim 1 wherein the method further comprises automatically validating the accuracy and completeness of the operative note.

5. The method of claim 4 wherein the method further comprises utilizing data related to the automatic validation to train the AI application via reinforcement learning.

6. The method of claim 1 wherein the method further comprises:
soliciting user feedback to validate the accuracy and completeness of the operative note; and
receiving the user feedback.

7. The method of example 6 wherein the method further comprises utilizing the user feedback to train the AI application via reinforcement learning.

8. The method of claim 1 wherein the surgical procedure data comprises multiple modalities of data.

9. The method of claim 1 wherein the surgical procedure data comprises intraoperative video data of the surgical procedure.

10. The method of claim 1 wherein the surgical procedure is a spinal surgical procedure.

11. A system for generating an operative note for a surgical procedure, the system comprising:
a sensor array including multiple sensors configured to capture surgical procedure data of the surgical procedure; and
an operative note generation device programmed with non-transitory computer readable instructions that, when executed by the operative note generation device, cause the operative note generation device to—
acquire the surgical procedure data captured by the sensor array;
identify features in the surgical procedure data relevant to the surgical procedure;
process the identified features to provide contextual information about the surgical procedure; and
utilize an artificial intelligence (AI) application to generate the operative note based on the identified features and the contextual information, wherein the operative note includes a natural language description of the surgical procedure.

12. The system of claim 11 wherein the operative note generation device is positioned local to the sensor array.

13. The system of claim 11 wherein the operative note generation device is positioned remote from the sensor array.

14. The system of claim 11 wherein the multiple sensors include RGB cameras, and wherein the surgical procedure data comprises RGB image data.

15. The system of claim 11 wherein the computer readable instructions, when executed by the operative note generation device, cause the operative note generation device to acquire the surgical procedure data in real time or near real time from the sensor array.

16. The system of claim 11 wherein the computer readable instructions, when executed by the operative note generation device, further cause the operative note generation device to:
acquire additional data related to the surgical procedure from a source other than the sensor array; and
process the identified features and the additional data to provide contextual information about the surgical procedure.

17. The system of claim 16 wherein the additional data comprises preoperative image data of a patient undergoing the surgical procedure.

18. The system of claim 11 wherein the computer readable instructions, when executed by the operative note generation device, further cause the operative note generation device to embed a hyperlink in the operative note, wherein the hyperlink links a textual description in the operative note to a corresponding feature identified in the surgical procedure data.

19. The system of claim 18 wherein the corresponding feature identified in the surgical procedure comprises a video segment.

20. A method of generating an operative note for a surgical procedure, the method comprising:
capturing surgical procedure data of the surgical procedure with a sensor array positioned to view the surgical procedure;
identifying features in the surgical procedure data relevant to the surgical procedure;
processing the identified features to provide contextual information about the surgical procedure; and
utilizing an artificial intelligence (AI) application to generate the operative note based on the identified features and the contextual information, wherein the operative note includes a natural language description of the surgical procedure.
